(19) United States
(12) Patent Application Publication
(10) Pub. No.: US 2007/0161645 A1
(43) Pub. Date: Jul. 12, 2007

(54) Thiazole inhibitors targeting resistant kinase mutations

(75) Inventors: Glenn Noronha, Oceanside, CA (US); Jianguo Cao, San Diego, CA (US); Binqi Zeng, San Diego, CA (US); Chi Ching Mak, San Diego, CA (US); Andrew McPherson, San Diego, CA (US); Joel Renick, San Diego, CA (US); Ved P. Pathak, San Diego, CA (US); Chun Chow, San Diego, CA (US); Moorthy Palanki, Encinitas, CA (US); Richard M. Soll, San Diego, CA (US); Daniel L. Lohse, San Diego, CA (US); John D. Hood, San Diego, CA (US); Elena Dneprovskaia, San Diego, CA (US)

Correspondence Address: DLA PIPER US LLP, 4365 EXECUTIVE DRIVE, SUITE 1100, SAN DIEGO, CA, 92121-2133, US

(73) Assignee: TargeGen, Inc., San Diego, CA (US)

(21) Appl. No.: 11/591,252

(22) Filed: Oct. 31, 2006

Related U.S. Application Data
(60) Provisional application No. 60/733,115, filed on Nov. 2, 2005.

(51) Int. Cl. A61K 31/506; A61K 31/496; A61K 31/454; A61K 31/427; A61K 31/428; C07D 417/14

(52) U.S. Cl. 514/252.19; 514/254.02; 514/326; 514/365; 544/295; 544/369; 546/208; 548/190

(57) ABSTRACT

A compound is provided, having the general structure (A): 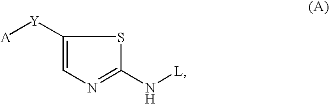 wherein A is an aryl or heteroaryl group, Y is a hydrophbic linking moiety, and L is a substitutent. The compound (A) can be used for treatment of various angiogenic-associated or hematologic disorders, such as myeloproliferative disorders in patients who do not respond to kinase-inhibition therapy that comprises administering currently used medications.

### CROSS REFERENCE TO RELATED APPLICATIONS

This application claims the benefit of priority under 35 U.S.C. § 119(e) of U.S. Ser. No. 60/733,115 filed Nov. 2, 2005, the entire content of which is incorporated herein by reference.

### BACKGROUND

1. Field of Invention

The present invention relates to the field of inhibitors of protein tyrosine kinases, their pharmaceutically acceptable compositions comprising the compounds of the invention and the methods of using the compositions in the treatment of various disorders. In particular, the present invention relates to several kinase inhibitors that can access residues deep within the hydrophobic pockets of kinases, or access portions of a conserved aspartic acid-phenylalanine-glycine (DFG) loop adjacent to the hydrophobic pockets of kinases, or circumvent the gatekeeper mutation.

2. Background of the Invention

Drug treatment induced resistance is an emerging theme of great importance in the design of inhibitors targeting various important human disease states. For example, Imatinib mesylate (Gleevec, ST1571) has become the standard of care for the treatment of patients with chronic myeloid leukemia (CML). Although responses in the chronic phase tend to be durable, relapse after an initial response is common in patients with more advanced disease. Point mutations within the kinase domain (KD) of BCR-ABL are the most common mechanism of acquired drug resistance, found in 50% to 90% of such patients.

These kinds of resistances are seen in other inhibitors of BCR-ABL used to treat CML, and in Gleevec resistant CML, as seen in the cases of Nilotinib (Tasigna, AMN-107) and Dasatinib (Sprycel, BMS-354825). Neither of these 2^{nd} generation, or follow-on compounds targets the kinase with the gatekeeper mutation.

Similar cases of resistance are seen in using Gleevec against other disease states, where different kinases are targeted by Gleevec—platelet-derived growth factor (PDGFR) for example. And resistance to other kinase inhibitors that are currently used therapies is an emerging theme, as seen in the cases drug induced resistances to gefinib (Iressa) and erlotinib (Tarceva) that are used to target epidermal growth factor receptor (EGFR). Both of these inhibitors are currently used therapies. Fairly common to all of these inhibitors is the inability of the inhibitors to target the kinase domain in the cases of the targeted protein having the so-called gatekeeper mutation.

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation or de-regulation, as well as over- or under-production of growth factors or cytokines has been implicated in many diseases, including but not limited too cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, survival, apoptosis, mitogenesis, cell cycle control, and cell mobility implicated in the aforementioned diseases.

The protein kinase super family of enzymes has emerged as an important class of targets for therapeutic intervention with small molecules due to dysregulated kinase activity in many pathological conditions including cancer. For example, Gleevec is the first protein tyrosine kinase inhibitor to be currently used for the treatment of human malignancy by virtue of its inhibition of several tyrosine kinases such as ABL, KIT, and PDGFR. Treatment with Gleevec as a single agent has demonstrated remarkable clinical efficacy in CML. The tyrosine kinase EGFR has been targeted with small molecule inhibitors such as Tarceva and Iressa for the treatment of patients with non-small cell lung carcinoma (NSCLC). SU 11248 (Sutent) is currently used for the treatment of certain tumors through its multi-modal action on the tyrosine kinases including the vascular endothelial growth factor receptor (VEGFR), KIT, and PDGFR. Inhibition of other kinases with small molecule inhibitors include the tyrosine kinase FLT3 that is expressed on blasts in most cases of acute myeloid leukemia (AML), the tyrosine kinases FGFR1, FGFR3, c-FMS, JAK, and SYK in a range of malignant hematological disorders, and ALK, c-met, and RET in a host of solid tumors.

Kinases other than tyrosine kinases are targets of small molecule inhibitors. For example, BAY 43-9006 (Sorafenib) exhibits inhibition of the serine threonine kinase RAF for the treatment of solid tumor malignancies, as well as the tyrosine kinase, VEGFR. The lipid kinase P13K is a potential kinase target for therapeutic intervention in a host of human cancers including colon, brain, breast, prostate, glioblastoma, melanoma, and endometrial carcinoma.

Inhibiting kinases with ATP-competitive kinase inhibitors blocks enzymatic activity of the kinases. Often treatment therapies result in drug resistance over a period of time. Quite often, drug resistance is largely on account of mutations that occur to prevent the pressures exerted by drug binding. Thus, despite success with Gleevec to treat CML through inhibition of the oncogene BCR-ABL, clinical resistance to the drug has been observed. Of the multiple mechanisms of drug resistance, mutations of the BCR-ABL kinase have been particularly problematic with 50-90% of the resistance to Gleevec arsing from mutations in the kinase domain.

A variety of the 2^{nd} generation agents mentioned above, such as Nilotinib, and Dasatinib are able to inhibit a large number of clinically relevant mutations. However, neither of these agents inhibits the T315I mutation, also known as the gatekeeper mutation, although this mutation is the largest singly occurring mutation to Gleevec mono-therapy, the current standard of care for CML. Mutation of the gatekeeper residue enables the protein to bind ATP and continue to function, while Gleevec is selectively rejected since it makes use of a hydrophobic pocket close to the ATP binding site, which ATP does not utilize. As a matter of fact, almost all small molecule inhibitors that are ATP-competitive utilize this hydrophobic pocket to attain much higher potency over ATP. Gleevec is no exception. It is therefore, not surprising that the gatekeeper and its mutation across numerous kinases is well known since most small molecule inhibitors of kinases are ATP competitive. Mutation of the gatekeeper residue confers resistance of kinases such as p38, SRC, EGFR to different ATP-competitive inhibitors, including SB203580, PP1, and PD153035, respectively. While mutations seem to be selectively enhanced under pressures from inhibitor molecules, the common theme of resitance to inhibitor molecules that serve as drugs is clearly emerging.

Preclinical and clinical data obtained to date suggest that, for human CML that is driven by BCR-ABL, the apparent ability of Dasatinib to bind to both the active and the inactive conformations of BCR-ABL affords this agent greater therapeutic potential compared with an agent, such as Gleevec, which binds only to an inactive form of the enzyme. Phase I and II clinical experience has shown promising results for Dasatinib in patients with Imatinib-resistant and Imatinib-intolerant disease. Current studies in human patients reveal that the potency and favorable profile of Dasatinib against wild-type ABL and several imatinib resistant ABL mutants is at least partially due to its ability to recognize multiple states of BCR-ABL. Yet, Dasatinib is till completely ineffective against the gatekeeper mutation, the single largest mutation arising from all existing therapies in CML.

The concept of using combination therapies to treat resistance to existing therapies makes use of the idea of different inhibitors exploring differing spaces and differing activation states of the kinases. Thus, low doses of 2^{nd} generation agents, Dasatinib or Nilotinib separately, or Dasatinib combined with low doses of Nilotinib may effectively suppress the emergence of almost all other mutations other than T315I. Because the nonhematologic side effects of Nilotinib and Dasatinib are not identical, patients with intolerance to either agent could be managed with combinations at low doses, avoiding toxicity while maintaining full antileukemic activity. Clearly, with T315I emerging as the mutation that is not targeted by any of these agents, an inhibitor of T315I is needed for patients that show resistance to all of these existing therapies.

A common structural theme, amongst kinases is the existence of particular pockets that are accessed by kinase inhibitors in both active and inactive states of the enzyme. Unlike ATP, which binds to the active site of all kinases, many small molecule kinase inhibitors derive their unusual potencies and specificities to particular pockets that are available to the inhibitor upon binding in addition to binding at ATP-binding residues. For example, the dual SRC and ABL inhibitor Dasatinib binds to this deep hydrophobic pocket defined by the protein in both SRC and ABL and does not form any key hydrogen bonding interactions within this deep specificity pocket. The gate-keeper residue, as the name describes, sits just at the entrance to this pocket and thus resistance to these inhibitors in large part, is conferred simply by mutations, especially at the gatekeeper residue. Dasatinib is completely un-effective against mutation of the gatekeeper T315I mutation.

Deep within the hydrophobic pocket is an acceptor residue, a glutamatic acid, forming a key salt bridge with a lysine (K295 in the case of SRC and K271 in the case of ABL). Other residues in close proximity are the Aspartic acid from the DFG portion of the activation loop and other conserved residues that are part of the activation mechanisms of these kinases. Despite their promixity and well-conserved nature across all kinases, kinase inhibitor design has failed in taking advantage of any of these key residues in any specific and targeted manner.

Clearly, with gatekeeper mutation resistance emerging in CML as the major mutation with combined and individual second generation therapies, an inhibitor of T315I remains an unmet need in CML. Currently used inhibitors targeting CML and other disease states do not describe specific designs to make use of residues deep within and adjacent to the hydrophobic pockets in the kinase domains. Designs targeting the gatekeeper resistant proteins are not described with currently used inhibitor series. Designs targeting the gatekeeper resistant proteins by targeting residues deep within and adjacent to the hydrophobic pockets in the kinase domains are not described for currently used inhibitors. The concept of inhibitor design and examples targeting conserved yet uniquely positioned residues deep within and proximal to the hydrophobic pocket as a part of inhibitor design to circumvent the gatekeeper mutation is provided here.

This concept is the basis for the inhibitors targeting the gatekeeper mutation in CML, where resistance is seen to all the current therapies including Gleevec, Sprycel and Tasigna, or any other inhibitor that does not target the gatekeeper mutation resistant ABL or BCR-ABL protein effectively. The concept can be applied in designing inhibitors that bind other kinases with gatekeeper mutations, where mutations in the gatekeeper residue arise on treatment with Gleevec, Sprycel and Tasigna, when these inhibitors are used to target these kinases, and such resistance is manifested rendering these inhibitors less effective or ineffective.

This concept can be applied in other kinds of drug related resistance as in the case of gatekeeper mutation resistance kinases from Tarceva, Iressa, and all other currently used kinase inhibitors that are currently used as therapies for other treatment conditions.

### DETAILED DESCRIPTION

A. Terms and Definitions.

The following terminology and definitions apply as used in the present application, generally in conformity with the terminology recommended by the International Union of Pure and Applied Chemistry (IUPAC):

The term “heteroatom” refers to any atom other than carbon, for example, N, O, or S.

The term “aromatic” refers to a cyclically conjugated molecular entity with a stability, due to delocalization, significantly greater than that of a hypothetical localized structure, such as the Kekule structure.

The term “heterocyclic,” when used to describe an aromatic ring, refers to the aromatic rings containing at least one heteroatom, as defined above.

The term “heterocyclic,” when not used to describe an aromatic ring, refers to cyclic (i.e., ring-containing) groups other than aromatic groups, the cyclic group being formed by between 3 and about 14 carbon atoms and at least one heteroatom described above.

The term “substituted heterocyclic” refers, for both aromatic and non-aromatic structures, to heterocyclic groups further bearing one or more substituents described below.

The term “alkyl” refers to a monovalent straight or branched chain hydrocarbon group having from one to about 12 carbon atoms, for example, methyl, ethyl, n-propyl, iso-propyl, n-butyl, iso-butyl, tert-butyl, n-pentyl (also known as n-amyl), n-hexyl, and the like. The term “lower alkyl” refers to alkyl groups having from 1 to about 6 carbon atoms.

The term “substituted alkyl” refers to alkyl groups further bearing one or more substituents such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, aldehyde, acyl, oxyacyl, carboxyl, sulfonyl, sulfonamide, sulfuryl, and the like.

The term “alkenyl” refers to straight-chained or branched hydrocarbyl groups having at least one carbon-carbon double bond, and having between about 2 and about 12 carbon atoms, and the term “substituted alkenyl” refers to alkenyl groups further bearing one or more substituents described above.

The term “alkynyl” refers to straight-chained or branched hydrocarbyl groups having at least one carbon-carbon triple bond, and having between about 2 and about 12 carbon atoms, and the term “substituted alkynyl” refers to alkynyl groups further bearing one or more substituents described above.

The term “aryl” refers to aromatic groups having between about 5 and about 14 carbon atoms and the term “substituted aryl” refers to aryl groups further bearing one or more substituents described above.

The term “heteroaryl” refers to aromatic rings, where the ring structure is formed by between 3 and about 14 carbon atoms and by at least one heteroatom described above, and the term “substituted heteroaryl” refers to heteroaryl groups further bearing one or more substituents described above.

The term “alkoxy” refers to the moiety —O-alkyl, wherein alkyl is as defined above, and the term “substituted alkoxy” refers to alkoxy groups further bearing one or more substituents described above.

The term “cycloalkyl” refers to alkyl groups having between 3 and about 8 carbon atoms arranged as a ring, and the term “substituted cycloalkyl” refers to cycloalkyl groups further bearing one or more substituents described above.

The term “alkylaryl” refers to alkyl-substituted aryl groups and the term “substituted alkylaryl” refers to alkylaryl groups further bearing one or more substituents described above.

The term “arylalkyl” refers to aryl-substituted alkyl groups and the term “substituted arylalkyl” refers to arylalkyl groups further bearing one or more substituents described above.

The term “arylalkenyl” refers to aryl-substituted alkenyl groups and the term “substituted arylalkenyl” refers to arylalkenyl groups further bearing one or more substituents described above.

The term “arylalkynyl” refers to aryl-substituted alkynyl groups and the term “substituted arylalkynyl” refers to arylalkynyl groups further bearing one or more substituents described above.

The term “arylene” refers to divalent aromatic groups having between 5 and about 14 carbon atoms and the term “substituted arylene” refers to arylene groups further bearing one or more substituents described above.

The term “kinase” refers to any enzyme that catalyzes the addition of phosphate groups to a protein residue; for example, serine and threonine kinases catalyze the addition of phosphate groups to serine and threonine residues.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “antibody” refers to intact molecules of polyclonal or monoclonal antibodies, as well as fragments thereof, such as Fab and F(ab′)₂, Fv and SCA fragments which are capable of binding an epitopic determinant.

### B. EMBODIMENTS OF THE INVENTION

According to an embodiment of the invention are provided for treatment of various diseases, disorders, and pathologies, including treatment of angiogenic-associated disorders, such as myeloproliferative disorder. The compounds include an aryl or a heteroaryl moiety and a hydrophobic linking moiety connecting the aryl or heteroaryl moiety to a thiazole-derived moiety.

The aryl or heteroaryl moiety carries a first substitutent comprising an acidic proton, such as hydroxyl, carboxyl, amino, or amido group, which can be attached to any position of the aryl or heteroaryl moiety as chemically reasonable. The thiazole-derived moiety carries a second substitutent comprising an amino group.

Schematically, therefore, the compounds of the present invention can be represented as the general structure (A): 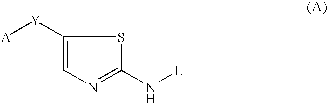
wherein A is an aryl or heteroaryl group, as discussed below, Y is a hydrophobic linking moiety, as discussed below, and L is: 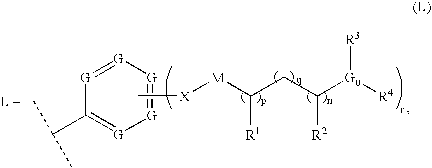
wherein each of the groups G can be independently N, CH, or C linked to X, where X can be any of a bond, O, C═O, SO₂, or CH₂ and M can be a bond or NR⁹; or X and M taken together can be a bond. Further, in the structure (A) each of R¹ and R² can be any of H, CF₃, F, Cl, Br, I, OH, OCH₃, CN, OCF₃, NH₂, C₁-C₆ substituted or unsubstituted alkyl, substituted or unsubstituted cycloalkyl, substituted or unsubstituted heterocycle, substituted or unsubstituted aryl, or substituted or unsubstituted heteroaryl; or R¹ and R² taken together can be a bond; or R¹ and R² taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ, wherein each of p, q, r, n, m is idependently an integer having the value between 0 and 6.

Further, in the structure (A) R⁹ can be one of H, C₁-C₆ substituted or unsubstituted alkyl, C₁-C₆ substituted or unsubstituted alkenyl, C₁-C₆ substituted or unsubstituted alkynyl, C₁-C₆ substituted or unsubstituted hydroxyalkyl or aminoalkyl, C₁-C₆ substituted or unsubstituted branched alkyl, C₁-C₆ substituted or unsubstituted cycloalkyl, substituted or unsubstituted aryl connected through carbon or a heteroatom, substituted or unsubstituted heteroaryl connected through carbon or a heteroatom, C₁-C₆ alkoxy, a halogen, CF₃, —OCF₃, CHR³R⁴, SR³, SOR³, SO₂R³, SO₂NR³R⁴, SO₃R³,POR³, PO₂R³, PO₂NR³R⁴, PO₂CR³R⁴, PO₃R³, NR³R⁴, NO₂, CN, OH, CONR³R⁴,COR³, COOR³, NR³COR⁴, NR³CONR³R⁴, OCONR³R⁴, CSNR³R⁴, CSR³, NR³CSNR³R⁴, SCONR³R⁴, SCSNR³R⁴ or SCSNR³R⁴; G₀ can be one of N, O , H, of CH, with the proviso that if G₀ is N, then each of R³ and R⁴ can be one of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, or substituted or unsubstituted heteroaryl, or R³ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ.

There are some additional provisos further directed to G₀ in the structure (A). More specifically, if G₀ is N, then R¹ and R⁹ taken together can form a moiety such as one of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NR⁹—(CH₂)ₘ, or (CH₂)ᵣ—O—(CH₂)ₘ; or R¹ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R³ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ.

If in the structure (A) G₀ is O, then R³ can be one of H, CF₃, F, Cl, Br, I, OH, OCH₃, CN, OCF₃, NH₂, C₁-C₆ alkyl and C₁-C₆ substituted or unsubstituted hydroxyalkyl or aminoalkyl, substituted or unsubstituted branched alkyl, substituted or unsubstituted cycloalkyl, substituted heterocyclic connected through carbon or nitrogen, substituted or unsubstituted aryl, or substituted or unsubstituted heteroaryl connected through carbon or nitrogen, with no group R⁴; R¹ and R⁹ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R¹ and R³ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R³ taken together can form a moiety such as one of (CH₂)ₘ, (CH₂)ᵣ—S—CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ.

If in the structure (A), G₀ =CH, then each of R³ and R⁴ can be one of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, C₁-C₆ substituted or unsubstituted heterocycle connected through carbon or nitrogen, or substituted or unsubstituted heteroaryl connected through carbon or nitrogen, or R³ and R⁴ taken together can form a moiety such as one of (CHR⁹)ᵣ—(CHR⁹)ₘ—(CHR⁹)ₚ, (CHR⁹)ᵣ—S—(CHR⁹)ₘ, (CHR⁹)ᵣ—SO—(CHR⁹)ₘ, (CHR⁹)ᵣ—SO₂—(CHR⁹)ₘ, (CHR⁹)ᵣ—NR⁹—(CHR⁹)ₘ, or (CHR⁹)ᵣ—O—(CHR⁹)ₘ.

The aryl or heteroaryl moiety A shown in the structure (A), that can be used in the compounds of the invention can include such exemplary moieties as the moieities derived from benzene (e.g., benzene itslef, phenol, toluene, phenylmethanol, chlorophenol, fluorophenol, halogenated alkyl benzene, aniline, benzamide, benziamines, benzoate, pyrocatechol, benzimide, or benzenesulfonamide), or from indole, indoline, indene, indazole, imidazole, benzothiazole, pyrazole, pyridine, pyrrolopyridine, benzimidazole, imidazopyridine, benzoisoxazole, phenylimidazole, benzotriazole, tetrazole, or anisole.

Some examples of specific moieties that can represent an aryl or heteroaryl moiety A shown in the structure (A), can include, but are not limited to, one of the following moieties:

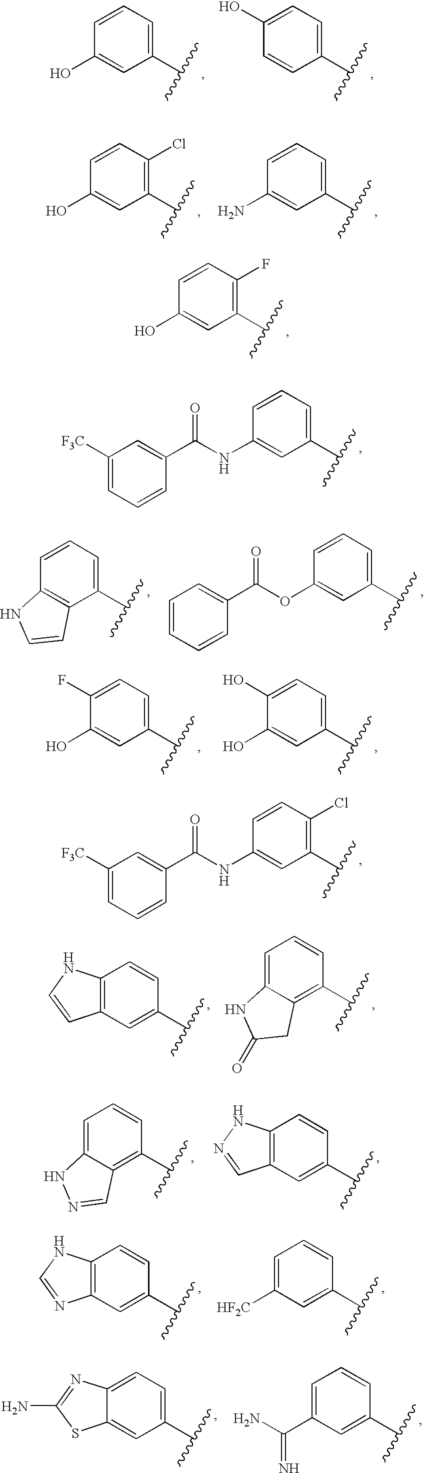

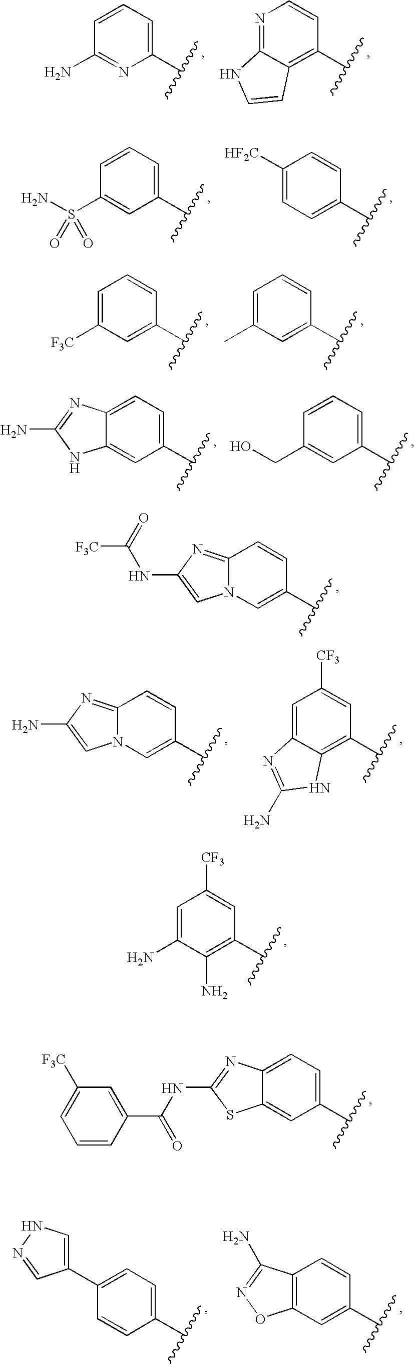

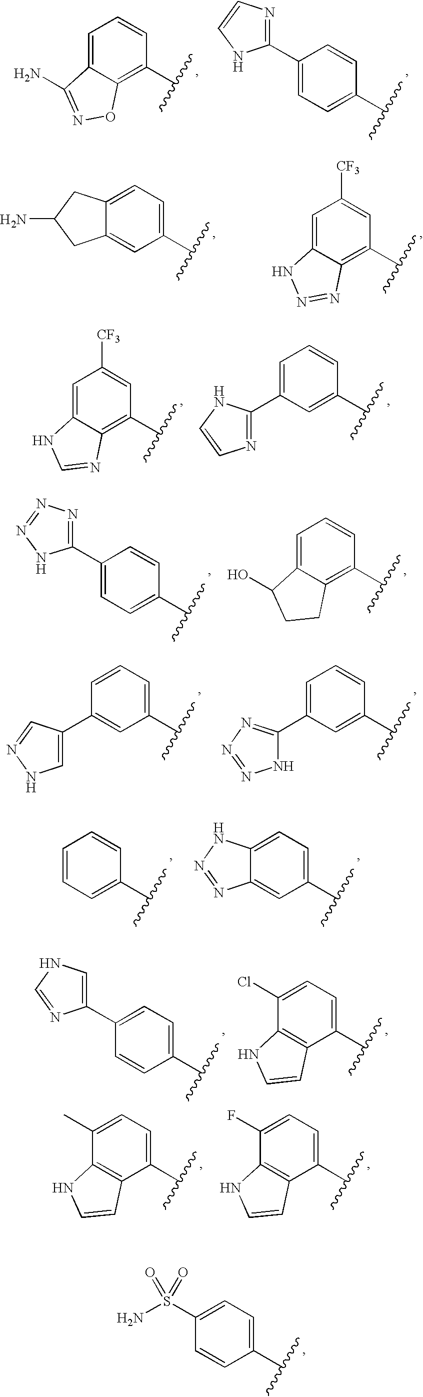

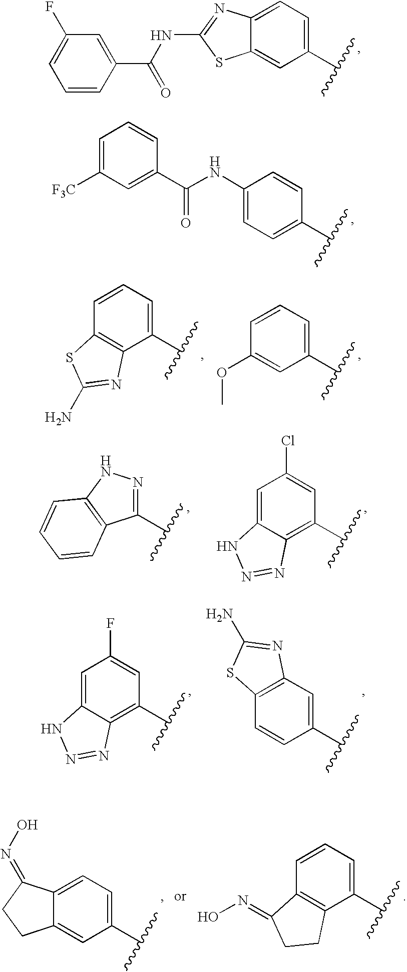

The above-mentioned linking moiety Y shown in the structure (A) can be attached to any position of the aryl or heteroaryl moiety A, and to any position of the thiazole-derived moiety, as chemically reasonable. The linking moiety Y that can be used includes an alkyl or an alkylene group, such as a group shown below:

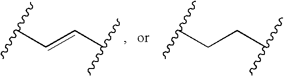

Some examples of specific moieties that can represent L include, but are not limited, to one of the following moieties

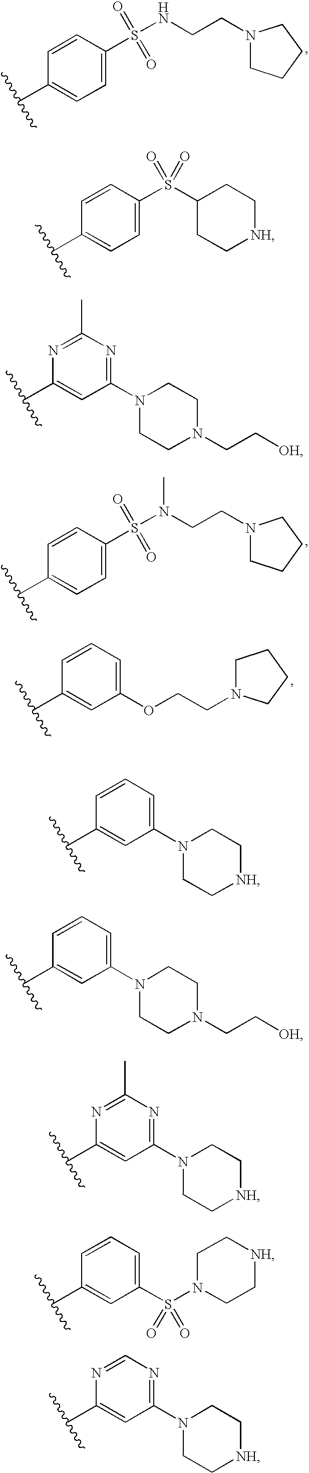

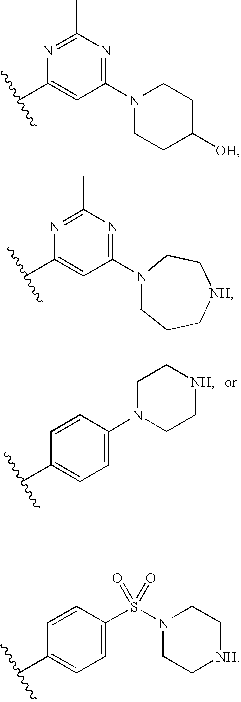

Some exemplary compounds described by structure (A) that can be used include, but are not limited to, the following compounds (I) through (XXIV) shown below:

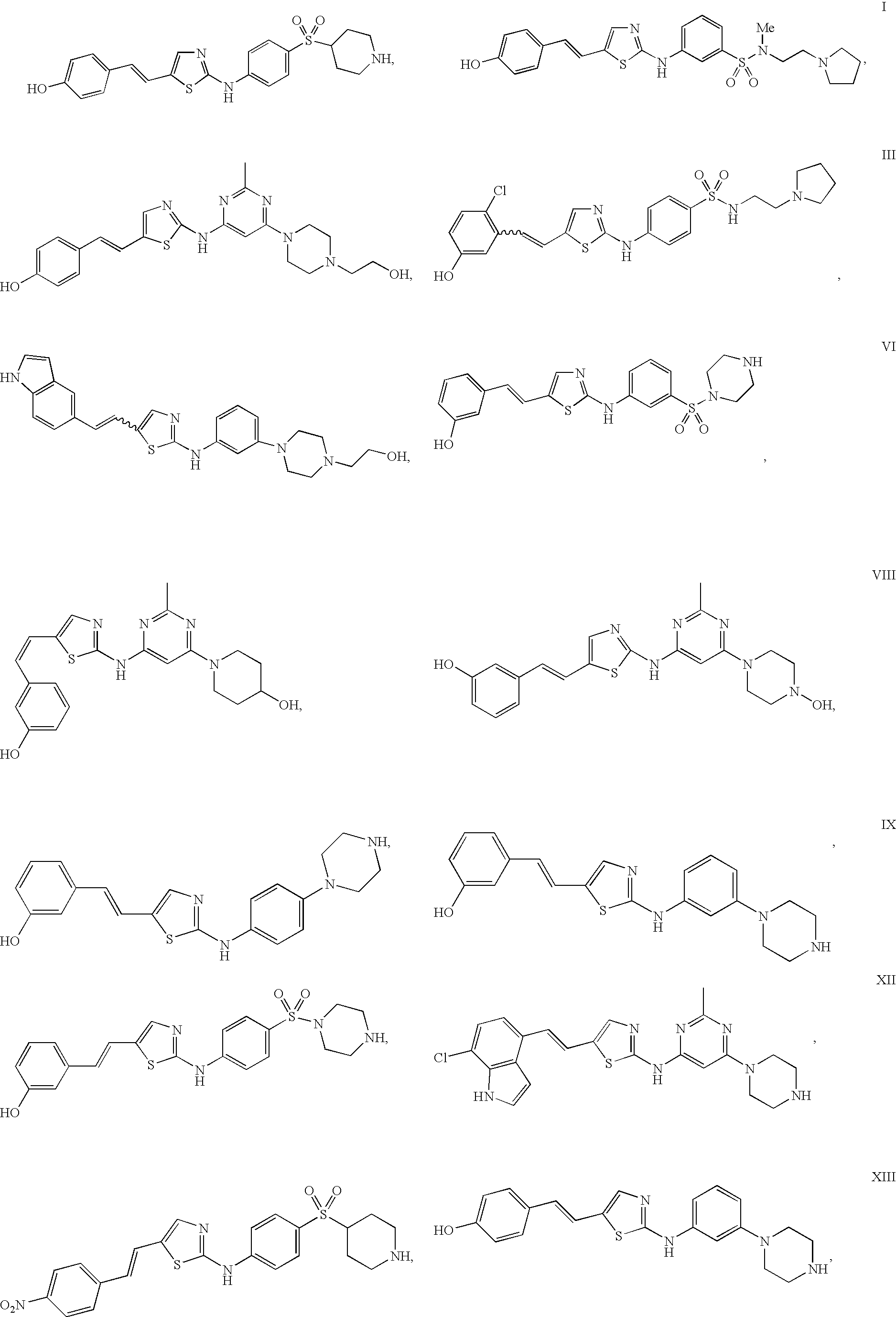

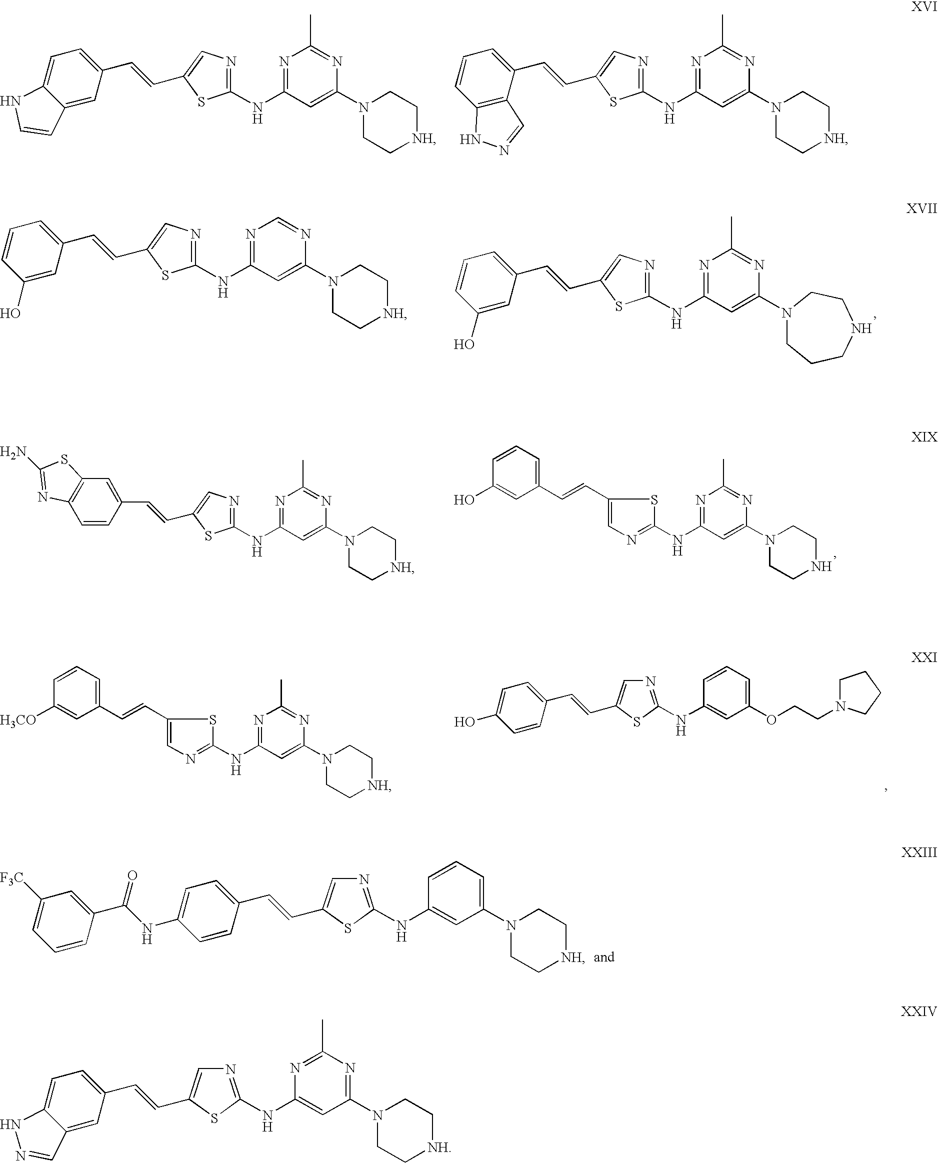

The compounds and methods of the present invention, or pharmaceutically acceptable salts, hydrates, solvates, crystal forms and individual diastereomers thereof, either when administered alone or in combination with other agents (e.g., chemotherapeutic agents or protein therapeutic agents described below) can be used for treating patients for whom traditional kinase-inhibition therapies with currently used medication are inefficient. The medication is defined as “currently used” if the medication is currently used for treatment of patients in need of treatment. Examples of such currently used medications include compounds (C), (D), or (E) shown below. Compound (C) is also known under the trade name GLEEVEC and is available from Novartis, and compound (D) is known by the trade name TASIGNA, and is available from Novartis, and (E) is known by the tradename SPRYCEL and is available from Bristol Myers Squibb.

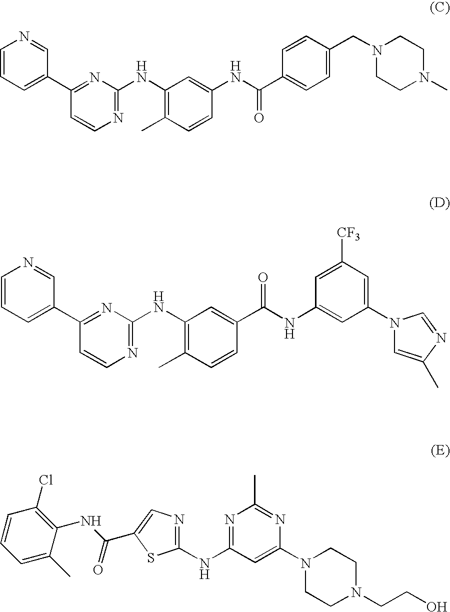

The inefficiency of the traditional kinase-inhibition treatments using compounds (C), (D), or (B) can be attributed to resistance the patients often develop to the treatment with these compound. The resistance can be caused by the kinase mutation, particularly the gatekeeper residue mutation. After the resistance has been developed, the traditional treatments (e.g., a GLEEVEC treatment of chronic myelogenous leukemia) no longer bring about sufficient therapeutic benefits. The therapy using a compound of the general structure (A) to replace all or a portion of the compounds (C), (D), or (E) can overcome the resistance and provide effective treatment.

Examples of disorders for treatment of which the compounds of structure (A), or pharmaceutically acceptable salts, hydrates, solvates, N-oxide(s), crystal forms and individual diastereomers thereof, can be used include, but are not limited to myeloproliferative disorders, proliferative diabetic retinopathy and other angiogenic-associated disorders including solid tumors and other types of cancer, eye disease, inflammation, psoriasis, and a viral infection. The kinds of cancer that can be treated include, but are not limited to, an alimentary/gastrointestinal tract cancer, colon cancer, liver cancer, skin cancer, breast cancer, ovarian cancer, prostate cancer, lymphoma, leukemia (including acute myelogenous leukemia and chronic myelogenous leukemia), kidney cancer, lung cancer, muscle cancer, bone cancer, bladder cancer or brain cancer.

Some examples of the diseases and disorders that can be treated also include ocular neovasculariaztion, infantile haemangiomas; organ hypoxia, vascular hyperplasia, organ transplant rejection, lupus, multiple sclerosis, rheumatoid arthritis, psoriasis, Type 1 diabetes and complications from diabetes, inflammatory disease, acute pancreatitis, chronic pancreatitis, asthma, allergies, adult respiratory distress syndrome, cardiovascular disease, liver disease, other blood disorders, asthma, rhinitis, atopic, dermatitits, autoimmune thryroid disorders, ulerative colitis, Crohn's disease, metastatic melanoma, Kaposi's sarcoma, multiple myeloma, conditions associated with cytokines, and other autoimmune diseases including glomerulonephritis, scleroderma, chronic thyroiditis, Graves' disease, autoimmune gastritis, autoimmune hemolytic anemia, autoimmune neutropenia, thrombocytopenia, atopy (e.g., allergic asthma, atopic dermatitis, or allergic rhinitis), chronic active hepatitis, myasthenia gravis, multiple sclerosis, inflammatory bowel disease, graft vs host disease, neurodegenerative diseases including motor neuron disease, Alzheimer's disease, Parkinson's disease, amyotrophic lateral scelerosis, Huntington's disease, cerebral ischemia, or neurodegenerative disease caused by traumatic injury, strike, gluatamate neurtoxicity or hypoxia; ischemic/reperfusion injury in stroke, myocardial ischemica, renal ischemia, heart attacks, cardiac hypertrophy, atherosclerosis and arteriosclerosis, organ hyoxia, and platelet aggregation.

Examples of some additional diseases and disorders that can be treated also include cell mediated hypersensitivity (allergic contact dermatitis, hypersensitivity pneumonitis), rheumatic diseases (e.g., systemic lupus erythematosus (SLE), juvenile arthritis, Sjogren's Syndrome, scleroderma, polymyositis, ankylosing spondylitis, psoriatic arthritis), viral diseases (Epstein Barr Virus, Hepatitis B, Hepatitis C, HIV, HTLV 1, Vaicella-Zoster Virus, Human Papilloma Virus), food allergy, cutaneous inflammation, and immune suppression induced by solid tumors.

Embodiments of the present invention also provide articles of manufacture that can include a packaging material and a pharmaceutical composition contained within the packaging material. The packaging material can comprise a label which indicates that the pharmaceutical composition can be used for treatment of one or more disorders identified above.

The pharmaceutical composition can include a compound according to the present invention. In addition to a compound of the present invention, the pharmaceutical may also contain other therapeutic agents, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques known in the art of pharmaceutical formulation.

Thus, in one embodiment, the invention provides a pharmaceutical composition including a therapeutic agent and a compound of the invention. The compound is present in a concentration effective to treat, for example, cancer or to treat another disease or disorder described above.

The compounds of the invention may be formulated into therapeutic compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like.

Salts of the invention can include amine salts formed by the protonation of an amino group with inorganic acids such as hydrochloric acid, hydrobromic acid, hydroiodic acid, sulfuric acid, phosphoric acid, and the like. Salts of the invention can also include amine salts formed by the protonation of an amino group with suitable organic acids, such as p-toluenesulfonic acid, acetic acid, methanesulfonic acid and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs of the invention compounds are included in the present invention.

Pharmaceutical compositions of the invention may be administered by any suitable means, for example, orally, such as in the form of tablets, capsules, granules or powders; sublingually; buccally; parenterally, such as by subcutaneous, intravenous, intramuscular, intrathecal, or intracistemal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The pharmaceutical compositions for the administration of the compounds of this embodiment, either alone or in combination with other therapeutic agents, may conveniently be presented in dosage unit form and may be prepared by any of the methods well known in the art of pharmacy. All methods include bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Also useful as a solubilizer is polyethylene glycol, for example. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a parenterally-acceptable diluent or solvent or cosolvent or complexing agent or dispersing agent or excipient or combination thereof, for example 1,3-butanediol, polyethylene glycols, polypropylene glycols, ethanol or other alcohols, provides, various brands of TWEEN surfactant, sodium dodecyl sulfate, sodium deoxycholate, dimethylacetamide, polysorbates, poloxamers, cyclodextrins, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In one embodiment, the invention compounds are administered in combination with an anti-inflammatory agent, antihistamines, chemotherapeutic agent, immunomodulator, therapeutic antibody or a protein kinase inhibitor, e.g., a tyrosine kinase inhibitor, to a subject in need of such treatment. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

The pharmaceutical composition and method of the present invention may further comprise other therapeutically active compounds as noted herein which are usually applied in the treatment of the above mentioned pathological conditions. Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8 gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

Other agents that may be administered in combination with invention compounds include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

When other therapeutic agents are employed in combination with the compounds of the present invention they may be used for example in amounts as noted in the Physician Desk Reference (PDR) or as otherwise determined by one having ordinary skill in the art.

In the treatment or prevention of conditions which involve cellular proliferation, an appropriate dosage level can generally be between about 0.01 and about 1000 mg per 1 kg of patient body weight per day which can be administered in single or multiple doses. For example, the dosage level can be between about 0.01 and about 250 mg/kg per day; more narrowly, between about 0.5 and about 100 mg/kg per day. A suitable dosage level can be between about 0.01 and about 250 mg/kg per day, between about 0.05 and about 100 mg/kg per day, or between about 0.1 and about 50 mg/kg per day, or about 1.0 mg/kg per day. For example, within this range the dosage can be between about 0.05 and about 0.5 mg/kg per day, or between about 0.5 and about 5 mg/kg per day, or between about 5 and about 50 mg/kg per day. For oral administration, the compositions can be provided in the form of tablets containing between about 1.0 and about 1,000 mg of the active ingredient, for example, about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1,000.0 mg of the active ingredient for the symptomatic adjustment of the dosage to the patient to be treated. The compounds can be administered on a regimen of 1 to 4 times per day, such as once or twice per day. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of the present invention can be used, alone or in combination with an effective amount of a therapeutic antibody (or therapeutic fragment thereof), a chemotherapeutic or an immunotoxic agent, for treatment of tumors. Illustrative examples of chemotherapeutic agents that can be used for this purpose include doxorubicin, docetaxel, or taxol. It should be further understood that the invention includes combination therapy including a compound of the invention, including but not limited to vasculostatic agents, such as tyrosine, serine or threonine kinase inhibitors, for example, and any chemotherapeutic agent or therapeutic antibody.

### C. EXAMPLES

The following examples are provided to further illustrate the advantages and features of the present invention, but are not intended to limit the scope of the invention.

### Example 1

### General Methods

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated. Analytical thin layer chromatography (TLC) was carried out on Merck Kieselgel 60 F₂₅₄ plates with visualization by ultraviolet and/or anisaldehyde, potassium permanganate or phosphomolybdic acid dips. Reverse-phase HPLC chromatography was carried out on Gilson 215 liquid handler equipped with Waters SymmetryShield™ RP18 7 μm (40×100 mm) Prep-Pak cartridge. Mobile phase consisted of standard acetonitrile (ACN) and DI Water, each with 0.1% TFA added. Purification was carried out at a flow rate of 40 mL/min. NMR spectra: ¹H Nuclear magnetic resonance spectra were recorded at 500 MHz. Data are presented as follows: chemical shift, multiplicity (s=singlet, d=doublet, t=triplet, q=quartet, qn=quintet, dd=doublet of doublets, m=multiplet, br s=broad singlet), coupling constant (J/Hz) and integration. Coupling constants were taken directly from the spectra and are uncorrected. Low resolution mass spectra: Electrospray (ES+) ionization was used. The protonated parent ion (M+H) or fragment of highest mass is quoted. Analytical gradient consisted of 10% ACN in water ramping up to 100% ACN over 5 minutes unless otherwise stated.

### Example 2

### 5-(4-Methoxystvryl)thiazol-2-amine (Intermediate 1)

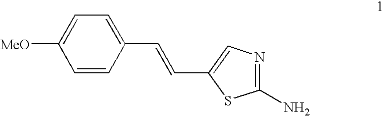

To a solution of 5-bromothiazol-2-amine hydrobromide (130 mg, 0.5 mmol) in 1,2-dimethoxyethane (DME, 4 mL) was added solution of (E)-2-(4-methoxyphenyl)vinylboronic acid (89 mg, 0.5 mmol) in EtOH (1 mL), solution of Na₂CO₃ (212 mg, 2.0 mmol) in H₂O (1 mL), and Pd(PPh₃)₄ (58 mg, 0.05 mmol). The mixture was heated at 110° C. for 15 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated until 5 mL remaining. Hexanes (100 mL) were added to the above solution and the solid collected by filtration. The title intermediate was used for next step without further purification.

### Example 3

### 4-((E)-2-(2-(4-(Piperidin-4-ylsulfonyl)phenylamino)thiazol-5-yl)vinyl)phenol (Compound I)

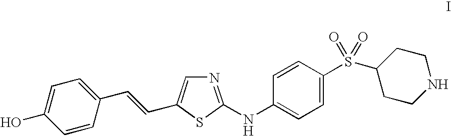

To a solution of intermediate 1 (0.12 g, 0.5 mmol) in 1,4-dioxane (20 mL) was added tert-butyl 4-(4-bromophenylsulfonyl)piperidine-1-carboxylate (295 mg, 0.73 mmol), Cs₂CO₃ (954 mg, 3.0 mmol), Pd₂(dba)₃ (65 mg, 0.07 mmol), and 4,5-bis(diphenylphosphino)-9,9-dimethyxanthene (Xant Phos, 122 mg, 0.21 mmol). The mixture was heated under reflux overnight under Ar. The solid was filtered off and the filtrate washed with brine (1×50 mL). The organic solution was separated and dried (Na₂SO₄). The solution was concentrated until 5 mL remaining and hexane (50 mL) was added, the solid was collected by filtration. The solid was dissolved in anhydrous CH₂Cl₂ (2mL) and the 1.0 M BBr₃ in CH₂Cl₂ (1.2 mL, 1.2 mmol) was added. The reaction was stirred for 2 h at room temperature. The saturated NaHCO₃ (20 mL) was added. The organic layer was separated and solid (containing product) collected by filtration. The aqueous was extracted with CH₂Cl₂ (2×10 mL). The combined organic layers was dried (Na₂SO₄) and the solvent was removed in vacuo. The residue was purified by HPLC to afford the title compound (6 mg of HCl salt, 2%) as a yellow solid.

¹H NMR (500 MHz, DMSO-d₆): δ 1.66-1.71 (m, 2H), 1.97-2.01 (m, 2H), 2.82-2.85 (m, 2H), 3.28-3.31 (m, 2H), 6.58 (d, J=15.9 Hz, 1H), 6.74 (d, J=7.9 Hz, 2H), 7.14 (d,J=15.9Hz, 1H), 7.34 (s, 1H), 7.35 (d, J=7.9Hz, 2H), 7.74 (d, J=8.4 Hz, 2H), 7.92 (d, J=8.4 Hz, 2H), 8.56 (br s, 1H), 9.13 (br s, 1H), 11.13 (br s, 1H). MS (ES+): m/z 442 (M+H)⁺.

### Example 4

### 3-Amino-N-methyl-N-(2-pyrrolidin-1-yl-ethyl)-benzenesulfonamide (Intermediate 2)

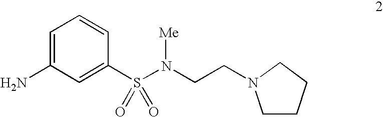

To a solution of the 3-nitrobenzene-1-sulfonyl chloride (2.0 g, 9 mmol) in anhydrous CH₂Cl₂ (100 mL) was added 2.0 M MeNH₂ in THF (18 mL, 36 mmol). The mixture was stirred overnight at room temperature. The saturated NaHCO₃ (100 mL) was added and the organic layer was separated. The aqueous was extracted with CH₂Cl₂ (2×30 mL). The combined organic layer was dried (Na₂SO₄) and concentrated in vacuo. The residue was dissolved in 1,4-dioxane (100-mL) followed by adding 1-(2-chloroethyl)pyrrolidine hydrochloride (1.7 g, 10 mmol) and Cs₂CO₃ (12 g, 36 mmol). The reaction mixture was heated under reflux overnight under Ar. The solid was filtered off and washed with EtOAc (200 mL).

The filtrated was washed with brine (1×150 mL). The organic layer was separated and aqueous was extracted with EtOAc (2×100 mL). The combined organic layer was dried and concentrated in vacuo. The residue was dissolved in MeOH (500 mL) and bubbled with Ar before adding 10% Pd—C. The reaction mixture was hydrogenated for 2 h. The catalyst was filtered off and washed with MeOH. The solvent was removed in vacuo to afford crude product (2.4 g, 94%) as a white solid.

### Example 5

### 5-(4-Methoxystyryl)-2-bromothiazole (Intermediate 3)

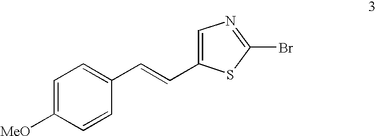

To a solution of (4-methoxy-benzyl)-triphenyl-phosphonium chloride (1.8 g, 4.0 mmol) in anhydrous DMF (30 mL) was added ^{t}BuOK (0.68 g, 6.0 mmol). The solution was stirred for 30 min before adding solution of 2-bromothiazole-5-carbaldehyde (0.69 g, 3.6 mmol) in DMF (10 mL). The mixture was stirred overnight. The solvent was removed in vacuo and the residue was suspended in EtOAc (100 mL) and washed with brine (2×50 mL). The aqueous was extracted with EtOAc (2×50 mL). The combined organic phase was dried (Na₂SO₄) and concentrated. The residue was purified by flash column (SiO₂/5% hexanes in EtOAc) to afford the title intermediate with cis (320 mg, 30%) and trans (200 mg, 19%) isomers.

¹H NMR (500 MHz, DMSO-d₆) (cis): δ 3.79 (s, 3H), 6.75 (d, J=6.5 Hz, 2H), 6.98 (d, J=8.7 Hz, 2H), 7.21 (d, J=8.7 Hz, 2H), 7.70 (s, 1H). ¹H NMR (500 MHz, DMSO-d₆) (trans): δ 3.32 (s, 3H), 6.93 (d, J=16.3 Hz, 1H), 6.95 (d, J=8.7 Hz, 2H), 7.30 (d, J=16.3 Hz, 1H), 7.52 (d, J=8.7 Hz, 2H), 7.68 (s, 1H).

### Example 6

### 3-{5-[2-(4-Hydroxy-phenyl)-vinyl]-thiazol-2-ylaminol}-N-methyl-N-(2-pyrrolidin-1-yl-ethyl)-benzenesulfonamide (Compound II)

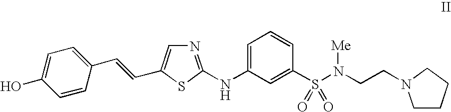

To a solution of 3 (100 mg, 0.34 mmol) in 1,4-dioxane (20 mL) was added 2 (87 mg, 0.34 mmol), Cs₂CO₃ (391 mg, 1.2 mmol), Pd₂(dba)₃ (27 mg, 0.03 mmol), and 4,5-bis(diphenylphosphino)-9,9-dimethyxanthene (Xant Phos, 52 mg, 0.09 mmol). The mixture was heated under reflux overnight under Ar. The solid was filtered off and the filtrate washed with brine (1×50 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) was added. The reaction was stirred for 2 h at room temperature. The saturated NaHCO₃ (20 mL) added. The organic layer was separated and solid (containing product) collected by filtration. The aqueous was extracted with CH₂Cl₂ (2×10 mL). The combined organic layer was dried (Na₂SO₄) and the solvent removed in vacuo. The residue was purified by HPLC to afford the title compound (12 mg of HCl salt, 2%) as a yellow solid.

¹H NMR (500 MHz, DMSO-d₆): δ 1.86-1.89 (m, 2H), 1.99 (br s, 2H), 2.76 (s, 3H), 3.04 (br s, 2H), 3.36 (br s, 4H), 3.56 (br s, 2H), 6.54 (d, J=16.0 Hz, 1H), 6.76 (d, J=8.5 Hz, 2H), 7.12 (d, J=16.0 Hz, 1H), 7.31 (s, 1H), 7.34 (d, J=8.5 Hz, 3H), 7.76 (t, J=8.0 Hz, 1H), 7.96 (d, J=8.0 Hz, 1H), 8.30 (s, 1H), 10.90 (br s, 1H), 11.22(br s, 1H). MS (ES+): m/z 485 (M+H)⁺.

### Example 7

### 2-(4-(6-Chloro-2-methylpyrimidin-4-yl)piperazin-1-yl)ethanol (Intermediate 4)

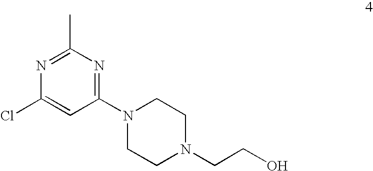

To a solution of 4,6-dichloro-2-methylpyrimidine (2.86 g, 17.5 mmol) in 1,4-dioxane (200 mL) was added 2-(piperazin-1-yl)ethanol (1.14 g, 1.08 mmol) and N,N-diisopropylethylamine (12.2 mL, 70 mmol). The reaction mixture was stirred overnight at room temperature. The solvent was removed in vacuo. The residue was dissolved in EtOAc (150 mL) and washed with saturated NaHCO₃ (2×50 mL). The organic layer was dried (Na₂SO₄). The solvent was removed in vacuo to afford the crude product (1.86 g, 83%) as yellow oil.

### Example 8

### 4-[2-(2-{6-[4-(2-Hydroxy-ethyl)-piperazin-1-yl]-2-methyl-pyrimidin-4-yl-amino}-thiazol-5-yl)-vinyl]-phenol (Compound III)

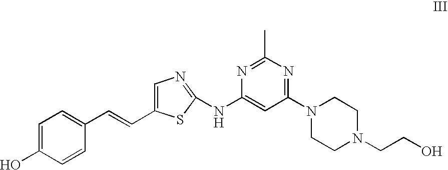

To a solution of intermediate 3 (116 mg, 0.5 mmol) in 1,4-dioxane (20 mL) was added intermediate 4 (128 mg, 0.5 mmol), Cs₂CO₃ (652 mg, 2.0 mmol), Pd₂(dba)₃ (46 mg, 0.05 mmol), and 4,5-bis(diphenylphosphino)-9,9-dimethyxanthene (Xant Phos, 87 mg, 0.15 mmol). The mixture was heated under reflux overnight under Ar. The solid was filtered off and the filtrate washed with brine (1×50 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added.

The organic layer was separated and solid (containing product) collected by filtration. The aqueous was extracted with CH₂Cl₂ (2×10 mL). The combined organic layer was dried (Na₂SO₄) and the solvent removed in vacuo. The residue was purified by HPLC to afford the title compound (2.5 mg of HCl salt, 1%) as a brown solid. MS (ES+): m/z 439 (M+H)⁺.

### Example 9

### 4-(5-Formyl-thiazol-2-ylamino)-N-(2-pyrrolidin-1-yl-ethyl)-benzensulfonamide (Intermediate 5)

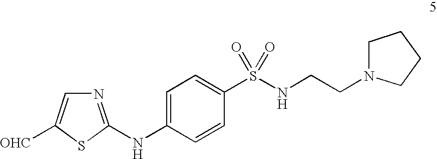

To a solution of 4-bromo-N-(2-pyrrolidin-1-yl-ethyl)-benzenesulfonamide (1.33 g, 4.0 mmol) in 1,4-dioxane (200 mL) was added 2-aminothiazole-5-carbaldehyde (0.51 g, 4.0 mmol), Cs₂CO₃ (5.2 g, 16.0 mmol), Pd₂(dba)₃ (0.37 g, 0.4 mmol), and 4,5-bis(diphenylphosphino)-9,9-dimethyxanthene (Xant Phos, 0.69 g, 1.6 mmol). The mixture was heated under reflux overnight under Ar. The solid was filtered off and the filtrate washed with brine (1×200 mL). The organic solution was separated and dried (Na₂SO₄). The solution was concentrated until 5 mL remaining and hexanes (50 mL) added, the solid was collected by filtration to afford the title intermediate (0.62 g, 41%) as a yellow solid.

### Example 10

### 4-{5-[2-(2-Chloro-5-hydroxy-phenyl)-vinyl]-thiazol-2-ylamino}-N-(2-pyrrolidin-1-yl-ethyl)-benzenesulfonamide (Compound IV)

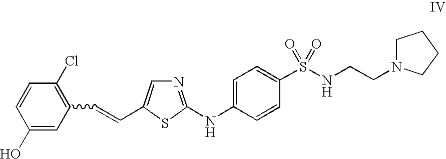

To a suspension of (2-chloro-5-methoxy-benzyl)-triphenyl-phosphonium bromide (250 mg, 0.5 mmol) in anhydrous THF (30 mL) was added NaH (200 mg, 5 mmol). The mixture was stirred for 1 h at room temperature before adding intermediate 5 (191 mg, 0.5 mmol). The mixture was heated under reflux overnight under Ar. The solid was filtered off and washed with THF. The filtrate was concentrated and residue was taken into EtOAc (100 mL) and washed with brine (2×50 mL). The combined organic layer was dried (Na₂SO₄) and the solvent removed in vacuo. The residue was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) collected by filtration. The aqueous was extracted with CH₂Cl₂ (2×10 mL). The combined organic layer was dried (Na₂SO₄) and the solvent in vacue. The residue was purified by HPLC to afford the title compound (3 mg of HCl salt, 1%) as a white solid. MS (ES+): m/z 505 (M+H)⁺.

### Example 11

### (5-Methyl-1H-indol-1-yl)(phenyl)methanone (Intermediate 6)

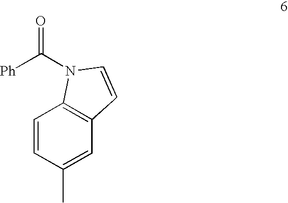

To a solution of the 5-methyl-1H-indole (2.6 g, 20 mmol) in anhydrous CH₂Cl₂ (100 mL) was added benzoyl chloride (2.5 mL, 22 mmol), DMAP (0.25 g, 2 mmol) and Et₃N (11.1 mL, 80 mmol). The mixture was stirred overnight at room temperature. The saturated NaHCO₃ (100 mL) was added and the organic layer separated. The aqueous was extracted with CH₂Cl₂ (2×30 mL). The combined organic layer was dried (Na₂SO₄) and concentrated in vacuo to afford the title intermediate (4.5 g, 96%) as a white solid.

### Example 12

### (5-(Bromomethyl)-1H-indol-1-yl)(phenyl)methanone (Intermediate 7)

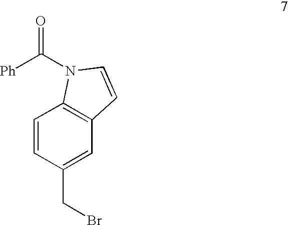

To a solution of intermediate 6 (4.5 g, 19.1 mmol) in CCl₄ (300 mL) was added N-bromo-succinimide (NBS, 4.1 g, 23 mmol) and 2,2′-azobisisobutyronitrile (AIBN, 0.32 g, 1.9 mmol). The mixture was heated under reflux for 4 h under Ar. The solid was filtered off and washed with CCl₄. The product was purified by flash column (SiO₂/20% EtOAc in hexanes) to afford the title intermediate as a white solid.

### Example 13

### (1Benzoyl-1H-indol-5-ylmethyl)-triphenyl-phosphonium bromide (Intermediate 8)

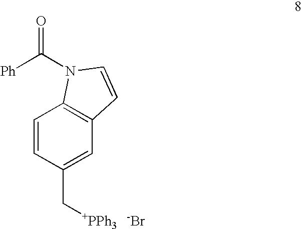

Intermediate 7 was dissolved in PhMe (200 mL) followed by adding Ph₃P (5.0 g, 19.1 mmol). The reaction was heated under reflux overnight. The solid was collected by filtration and washed with PhMe (2×50 mL) to afford the title intermediate (8.5 g, 77% in two steps) as a white solid.

### Example 14

### 5-(2-(2-Bromothiazol-5-yl)vinyl)-1H-indole (Intermediate 9)

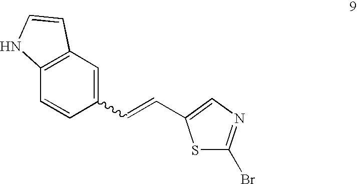

To a solution of intermediate 8 (1.16 g, 2 mmol) in anhydrous DMF (30 mL) was added ^{t}BuOK (0.34 g, 3.0 mmol). The solution was stirred for 30 min. before adding solution of 2-bromothiazole-5-carbaldehyde (0.39 g, 2.0 mmol) in DMF (10 mL). The mixture was stirred overnight. The benzoyl group was removed during the reaction. The solvent was removed in vacuo and the residue suspended in EtOAc (100 mL) and washed with brine (2×50 mL). The aqueous layer was extracted with EtOAc (2×50 mL). The combined organic phase was dried (Na₂SO₄) and concentrated. The residue was purified by flash column (SiO₂/5% hexanes in EtOAc) to afford the title intermediate as a mixture of cis and trans isomers (160 mg, 13%).

### Example 15

### 2-(4-(3-Bromophenyl)piperazin-1-yl)ethanol (Intermediate 10)

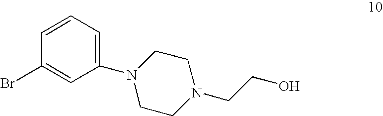

To a solution of 1-(3-bromophenyl)piperazine (1 g, 4.1 mmol) in DMF was added 2-bromoethanol (0.35 mL, 5 mmol) and K₂CO₃ (2.3 g, 17 mmol). The mixture was stirred overnight at room temperature. The solid was filtered off and washed with CH₂Cl₂. The filtrate was concentrated in vacuo. The residue was dissolved in CH₂Cl₂ (100 mL) and washed with brine (2×50 mL). The organic layer was collected and dried (Na₂SO₄). The solvent was removed in vacuo to afford the crude product for next step.

### Example 16

### 2-(4-(3-Aminophenyl)piperazin-1-yl)ethanol (Intermediate 11)

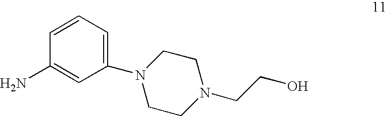

To a solution of intermediate 10 (1.33 g, 4.0 mmol) in 1,4-dioxane (200 mL) was added 2-aminothiazole-5-carbaldehyde (1.0 g, 3.5 mmol), Cs₂CO₃ (4.4 g, 13.5 mmol), Pd₂(dba)₃ (0.24 g, 0.3 mmol), and 4,5-bis(diphenylphosphino)-9,9-dimethyxanthene (Xant Phos, 0.52 g, 0.9 mmol). The mixture was heated under reflux overnight under Ar. The solid was filtered off and the filtrate washed with brine (1×200 mL). The organic solution was separated and dried (Na₂SO₄). The solvent was removed in vacuo. The residue was dissolved in MeOH (20 mL) followed by added 30% KOH (20 mL). The mixture was heated at 100° C. for 4 h. After reaction cool down, EtOAc (100 mL) was added and organic layer separated. The aqueous was extracted with EtOAc (2×50 mL). Combined organic layer was dried (Na₂SO₄). The solvent was removed to afford the title intermediate (0.41 g, 53%) as a black solid.

### Example 17

### 2-(4-(3-(5-((E)-2-(1H-indol-5-yl)vinyl)thiazol-2-ylamino)phenyl)piperazin-1-yl)ethanol (Compound V)

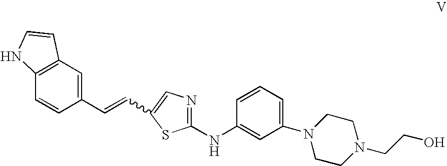

To a solution of intermediate 9 (134 mg, 0.44 mmol) in 1,4-dioxane (50 mL) was added (11) (98 mg, 0.44 mmol), Cs₂CO₃ (650 mg, 2.0 mmol), Pd₂(dba)₃ (46 mg, 0.05 mmol), and 4,5-bis(diphenylphosphino)-9,9-dimethyxanthene (Xant Phos, 87 mg, 0.15 mmol). The mixture was heated under reflux overnight under Ar. The solid was filtered off and the filtrate washed with brine (1×100 mL). The organic solution was separated and dried (Na₂SO₄). The solution was concentrated until 5 mL remaining and hexanes (50 mL) added, the solid was collected by filtration to afford the title compound (mixture of cis and trans isomers) as a yellow solid (4 mg, 2%). MS (ES+): m/z 446 (M+H)⁺.

### Example 18

### 5-(3-Methoxystyryl)-2-bromothiazole (Intermediate 12)

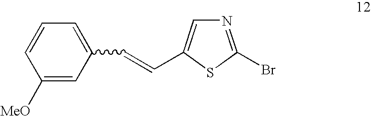

To a solution of (3-methoxy-benzyl)-triphenyl-phosphonium bromide (2.0 g, 4.8 mmol) in anhydrous DMF (30 mL) was added ^{t}BuOK (0.77 g, 6.9 mmol). The solution was stirred for 30 min before adding solution of 2-bromothiazole-5-carbaldehyde (0.77 g, 4.0 mmol) in DMF (10 mL). The mixture was stirred overnight. The solvent was removed in vacuo and the residue suspended in EtOAc (100 mL) and washed with brine (2×50 mL). The aqueous was extracted with EtOAc (2×50 mL). The combined organic phase was dried (Na₂SO₄) and concentrated. The residue was purified by flash column (SiO₂/40% hexanes in EtOAc) to afford the title intermediate (1.2 g, 98%) as a mixture of cis and trans isomers.

### Example 19

### 4-(3-Amino-benzenesulfonyl)-piperazine-1-carboxylic acid tert-butyl ester (Intermediate 13)

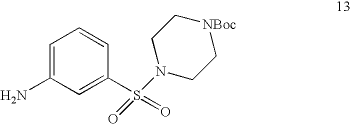

To a solution of tert-butyl piperazine-1-carboxylate (374 mg, 2.0 mmol) in CH₂Cl₂ (50 mL) was added 3-nitrobenzene-1-sulfonyl chloride (488 mg, 2.2 mmol), and Et₃N (1.12 mL, 8.0 mmol). The reaction mixture was stirred overnight at room temperature. The saturated NaHCO₃ (100 mL) was added. The organic layer was separated and aqueous extracted with CH₂Cl₂ (2×30 mL). The combined organic layer was dried (Na₂SO₄). The solvent was removed in vacuo. The residue was dissolved in MeOH (100 mL) and bubbled with Ar before adding 10% Pd—C (200 mg). The hydrogenation was taken 2 h. The catalyst was filtered off and washed with MeOH. The solvent was removed in vacuo to afford the crude product as a yellow solid.

### Example 20

### 3-(-{2-[3-(Piperazine-1-sulfonyl)-phenylamino]-thiazol-5yl}-vinyl)-phenol (Compound VI)

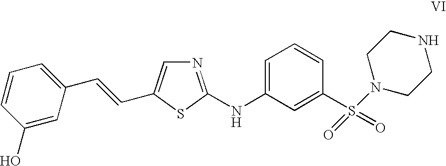

To a solution of intermediate 12 (220 mg, 0.74 mmol) in 1,4-dioxane (100 mL) was added 13 (253 mg, 0.74 mmol), Cs₂CO₃ (978 mg, 3.0 mmol), Pd₂(dba)₃ (65 mg, 0.07 mmol), and 4,5-bis(diphenylphosphino)-9,9-dimethyxanthene (Xant Phos, 122 mg, 0.21 mmol). The mixture was heated under reflux overnight under Ar. The solid was filtered off and the filtrate washed with brine (1×100 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) collected by filtration. The aqueous was extracted with CH₂Cl₂ (2×10 mL). The combined organic layer was dried (Na₂SO₄) and the solvent removed in vacuo. The residue was purified by HPLC to afford the title compound (11 mg of HCl salt, 3%) as a yellow solid.

¹H NMR (500 MHz, DMSO-d₆): δ 3.17 (br s, 8H), 6.55 (d, J=16.0 Hz, 1H), 6.66 (dd, J=7.9, 1.8 Hz, 1H), 6.90 (s, 1H), 6.95 (d, J=7.9 Hz, 1H), 7.14 (t, J=7.9 Hz, 1H), 7.28 (d, J=16.0 Hz, 1H), 7.33 (d, J=8.1 Hz, 1H), 7.42 (s, 1H), 7.62 (t, J=8.1 Hz, 1H), 7.94 (dd, J=8.3, 2.3 Hz, 1H), 8.27 (t, J=1.9 Hz, 1H), 9.08 (br s, 2H), 11.04 (br s, 1H). MS (ES+): m/z 443 (M+H)⁺.

### Example 21

### 1-(6-Chloro-2-methylpyrimidin-4-yl)piperidin-4-ol (Intermediate 14)

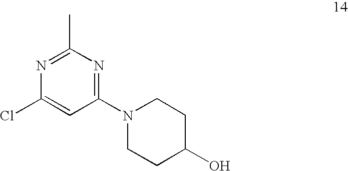

To a solution of 4,6-dichloro-2-methylpyrimidine (2.73 g, 16.7 mmol) in 1,4-dioxane (200 mL) was added piperidin-4-ol (1.13 g, 11.1 mmol) and N,N-diisopropylethylamine (7.9 mL, 45 mmol). The reaction mixture was stirred 72 h at room temperature. The solvent was removed in vacuo. The residue was dissolved in EtOAc (150 mL) and washed with saturated NaHCO₃ (2×50 mL). The organic layer was dried (Na₂SO₄). The solvent was removed in vacuo to afford the crude product (2.5 g, 98%) as yellow oil.

### Example 22

### 4-Chloro-2-methyl-6-(4-(tetrahydro-2H-pyran-2-yloxy)piperidin-1-yl)pyrimidine (Intermediate 15)

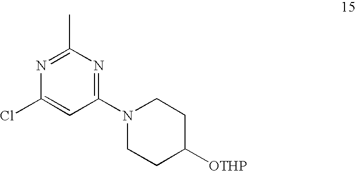

To a solution of intermediate 14 (456 mg, 2 mmol) in CH₂Cl₂ (100 mL) was added 3,4-dihydro-2H-pyran (0.36 mL, 4 mmol) and 4-methylbenzenesulfonic acid (PTSA, 76 mg, 0.4 mmol). The reaction mixture was stirred overnight at room temperature. Saturated NaHCO₃ solution (100 mL) was added and organic layer separated. The aqueous was extracted with CH₂Cl₂ (2×30 mL). The combined organic layer was dried (Na₂SO₄). The solvent was removed in vacuo to afford the crude product (760 mg, 94%) as yellow oil.

### Example 23

### 2-(2-Methyl-6-(4-(tetrahydro-2H-pyran-2-yloxy)piperidin-1-yl)pyrimidin-4-ylamino)thiazole-5-carbaldehyde (Intermediate 16)

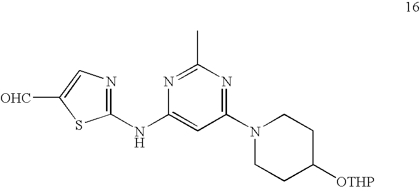

To a solution of intermediate 15 (470 mg, 1.5 mmol) in 1,4-dioxane (200 mL) was added 2-aminothiazole-5-carbaldehyde (193 mg, 1.5 Mmol), Cs₂CO₃ (1.95 g, 6.0 mmol), Pd₂(dba)₃ (137 mg, 0.15 mmol), and 4,5-bis(diphenylphosphino)-9,9-dimethyxanthene (Xant Phos, 260 mg, 0.45 mmol). The mixture was heated under reflux overnight under Ar. The solid was filtered off and the filtrate washed with brine (1×100 mL). The organic solution was separated and dried (Na₂SO₄). The solvent was removed in vacuo. The residue was purified by flash column (SiO₂/60% EtOAc in hexanes) to afford the title intermediate (600 mg, 98%) as yellow oil.

### Example 24

### cis-1-(6-{5-[2-(3-Hydroxy-phenyl)-vinyl]-thiazol-2-ylamino}-2-methyl-pyrimidin-4-yl)-piperidin-4-ol (Compound VII)

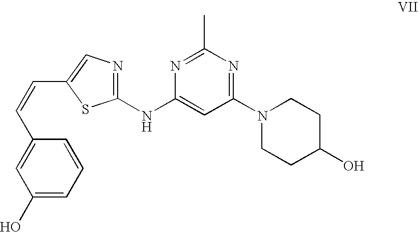

To a solution of (3-methoxy-benzyl)-triphenyl-phosphonium bromide (368 mg, 0.79 mmol) in anhydrous THF (30 mL) was added 2.5 M ^{t}BuLi in hexanes (0.5 mL, 1.25 mmol). The solution was stirred for 30 min before adding solution of intermediate 16 (320 mg, 0.79 mmol) in THF (10 mL). The mixture was stirred overnight. The solvent was removed in vacuo and the residue suspended in EtOAc (100 mL) and washed with brine (2×50 mL). The aqueous was extracted with EtOAc (2×50 mL). The combined organic phase was dried (Na₂SO₄) and concentrated. The residue was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) was collected by filtration. The aqueous was extracted with CH₂Cl₂ (2×10 mL). The combined organic layer was dried (Na₂SO₄) and the solvent removed in vacuo. The residue was purified by HPLC to afford the title compound (6 mg, 2%) as a yellow solid.

¹H NMR (500 MHz, DMSO-d₆): δ 1.73 (br s, 2H), 2.12 (br s, 2H), 2.55 (s, 3H), 3.05 (br s, 2H), 3.21 (br s, 2H), 4.63-4.67 (m, 1H), 6.71 (d, J=7.1 Hz, 1H), 7.02 (d, J=3.2 Hz, 1H), 7.11 (d, J=9.0 Hz, 1H), 7.20 (t, J=8.0 Hz, 1H), 7.33 (d, J=8.9 Hz, 1H), 7.45 (d, J=9.0 Hz, 1H), 7.72 (s, 1H), 9.10 (br s, 2H), 10.23 (s, 1H). MS (ES+): m/z 410 (M+H)⁺.

### Example 25

### trans-1-(6-{5-[2-(3-Hydroxy-phenyl)-vinyl]-thiazol-2-ylamino}-2-methyl-pyrimidin-4-yl)-piperidin-4-ol (Compound VIII)

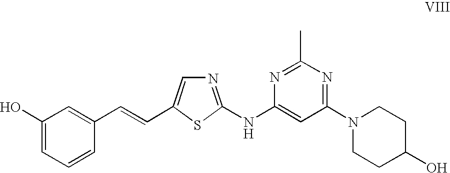

To a solution of (3-methoxy-benzyl)-triphenyl-phosphonium bromide (368 mg, 0.79 mmol) in anhydrous THF (30 mL) was added 2.5 M ^{t}BuLi in hexanes (0.5 mL, 1.25 mmol). The solution was stirred for 30 min before adding solution of intermediate 16 (320 mg, 0.79 mmol) in THF (10 mL). The mixture was stirred overnight. The solvent was removed in vacuo and the residue suspended in EtOAc (100 mL) and washed with brine (2×50 mL). The aqueous was extracted with EtOAc (2×50 mL). The combined organic phase was dried (Na₂SO₄) and concentrated. The residue was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) was collected by filtration. The aqueous was extracted with CH₂Cl₂ (2×10 mL). The combined organic layer was dried (Na₂SO₄) and the solvent removed in vacuo. The residue was purified by HPLC to afford the title compound (3 mg, 1%) as a yellow solid.

¹H NMR (500 MHz, DMSO-d₆): δ 1.42 (br s, 2H), 1.82 (br s, 2H), 2.55 (s, 3H), 3.50 (br s, 2H), 3.80 (br s, 2H), 4.63-4.67 (m, 1H), 6.70 (br s, 1H), 6.92 (s, 1H), 6.96 (d, J=7.7 Hz, 1H), 7.10-7.16 (m, 2H), 7.28 (d, J=16.2 Hz, 1H), 7.53 (s, 1H), 9.17 (br s, 1H), 10.28 (s, 1H). MS (ES+): m/z 410 (M+H)⁺.

### Example 26

### 3-((E)-2-(2-(4-(Piperazin-1-yl)phenylamino)thiazol-5-yl)vinyl)phenol (Compound IX)

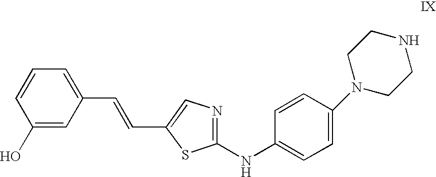

To a solution of intermediate 12 (200 mg, 0.67 mmol) in 1,4-dioxane (100 mL) was added tert-butyl 4-(4-aminophenyl)piperazine-1-carboxylate (187 mg, 0.67 mmol), Cs₂CO₃ (782 mg, 2.4 mmol), Pd₂(dba)₃ (55 mg, 0.06 mmol), and 4,5-bis(diphenylphosphino)-9,9-dimethyxanthene (Xant Phos, 104 mg, 0.18 mmol). The mixture was heated under reflux overnight under Ar. The solid was filtered off and the filtrate washed with brine (1×100 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) was collected by filtration. The aqueous was extracted with CH₂Cl₂ (2×10 mL). The combined organic layer was dried (Na₂SO₄) and the solvent removed in vacuo. The residue was purified by HPLC to afford the title compound (51 mg of HCl salt, 18%) as a white solid.

¹H NMR (500 MHz, DMSO-d₆): δ 3.23 (br s, 4H), 3.34-3.37 (m, 4H), 6.49 (d, J=16.0 Hz, 1H), 6.65 (dd, J=8.0, 2.2 Hz, 1H), 6.88 (s, 1H); 6.92 (d, J=7.9 Hz, 1H), 7.04 (d, J=9.1 Hz, 2H), 7.11 (t, J=7.9 Hz, 1H), 7.23 (d, J=16.0 Hz, 1H), 7.34 (s, 1H), 7.51 (d, J=9.1 Hz, 2H), 9.28 (br s, 1H), 10.77 (br s, 1H). MS (ES+): m/z 379 (M+H)⁺.

### Example 27

### tert-Butyl 4-(3-aminophenyl)piperazine-1-carboxylate (Intermediate 17)

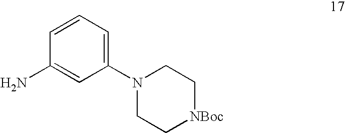

To a solution of 1-(3-nitrophenyl)piperazine (3.6 g, 15 mmol) in CH₂Cl₂ (100 mL) was added di-tert-butyl carbonate (5 g, 23 mmol) and N,N-dimethylpyridin-4-amine (0.37 g, 3 mmol). The mixture was stirred for 20 h at room temperature and saturated NaHCO₃ solution (100 mL) added. The organic layer was separated and aqueous extracted with CH₂Cl₂ (50 mL×2). The combined organic solution was dried and concentrated in vacuo. The residue was dissolved in MeOH and bubbled with Ar for 2 min. before adding 10% Pd—C. The hydrogenation was finished in 4 h. The catalyst was removed by filtration and solvent removed in vacuo to afford the title intermediate (4.01 g, 96%) as a white solid.

### Example 28

### 3-((E)-2-(2-(3-(Piperazin-1-yl)phenylamino)thiazol-5-yl)vinyl)phenol (Compound X)

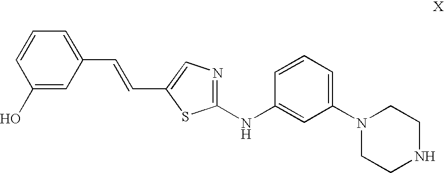

To a solution of intermediate 12 (140 mg, 0.47 mmol) in 1,4-dioxane (50 mL) was added intermediate 17 (131 mg, 0.47 mmol), Cs₂CO₃ (650 mg, 2.0 mmol), Pd₂(dba)₃ (46 mg, 0.05 mmol), and 4,5-bis(diphenylphosphino)-9,9-dimethyxanthene (Xant Phos, 90 mg, 0.15 mmol). The mixture was heated under reflux overnight under Ar. The solid was filtered off and the filtrate washed with brine (1×100 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) collected by filtration. The aqueous was extracted with CH₂Cl₂ (2×10 mL). The combined organic layer was dried (Na₂SO₄) and the solvent removed in vacuo. The residue was purified by HPLC to afford the title compound (29 mg of HCl salt, 15%) as a white solid.

¹H NMR (500 MHz, DMSO-d₆): δ 3.19 (br s, 4H), 3.35-3.37 (m, 4H), 6.50 (d, J=16.0 Hz, 1H), 6.64-6.68 (m, 2H), 6.88 (t, J=2.0 Hz, 1H), 6.93 (d, J=7.8 Hz, 1H), 7.08 (dd, J=7.8, 1.7 Hz, 1H), 7.13 (t, J=7.8 Hz, 1H), 7.21 (t, J=8.0 Hz, 1H), 7.25 (d, J=16.0 Hz, 1H), 7.34 (t, J=2.1 Hz, 1H), 7.35 (s, 1H), 9.20 (br s, 2H), 10.60 (br s, 1H). MS (ES+): m/z 379 (M+H)⁺.

### Example 29

### tert-Butyl 4-(4-aminophenylsulfonyl)piperidine-1-carboxylate (Intermediate 18)

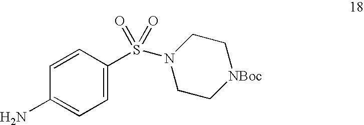

To a solution of the 4-nitrobenzene-1-sulfonyl chloride (2.7 g, 12 mmol) in anhydrous CH₂Cl₂ (100 mL) was added tert-butyl piperazine-1-carboxylate (1.8 g, 10 mmol), and Et₃N (5.6 mL, 40 mmol). The mixture was stirred overnight at room temperature. Saturated NaHCO₃ solution (100 mL) was added and the organic layer separated. The aqueous was extracted with CH₂Cl₂ (2×30 mL). The combined organic layer was dried (Na₂SO₄) and concentrated in vacuo. The residue was dissolved in MeOH (500 mL) and bubbled with Ar before adding 10% Pd—C. The reaction mixture was hydrogenated for 2 h. The catalyst was filtered off and washed with MeOH. The solvent was removed in vacuo to afford the crude product (3.2 g, 95%) as a white solid.

### Example 30

### 3-(-{2-[4-(Piperazine-1-sulfonyl)-phenylamino]-thiazol-5yl}-vinyl)-phenol (Compound XI)

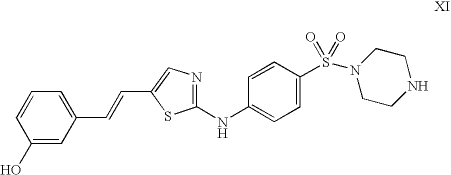

To a solution of intermediate 12 (220 mg, 0.74 mmol) in 1,4-dioxane (50 mL) was added intermediate 18 (253 mg, 0.74 mmol), Cs₂CO₃ (978 mg, 3.0 mmol), Pd₂(dba)₃ (65 mg, 0.07 mmol), and 4,5-bis(diphenylphosphino)-9,9-dimethyxanthene (Xant Phos, 122 mg, 0.21 mmol). The mixture was heated under reflux overnight under Ar. The solid was filtered off and the filtrate washed with brine (1×100 mL). The organic solution was separated and dried (Na₂SO₄). The solution was concentrated until 5 mL remaining and hexanes (50 mL) added, the solid was collected by filtration. The solid was dissolved in anhydrous CH₂Cl₂ (2 mL) and 1.0 M BBr₃ in CH₂Cl₂ (1.2 mL, 1.2 mmol) added. The reaction was stirred for 2 h at room temperature. Saturated NaHCO₃ solution (20 mL) was added. The organic layer was separated and solid (containing product) collected by filtration. The aqueous was extracted with CH₂Cl₂ (2×10 mL). The combined organic layer was dried (Na₂SO₄) and the solvent removed in vacuo. The residue was purified by HPLC to afford the title compound (15 mg of HCl salt, 4%) as a white solid.

¹H NMR (500 MHz, DMSO-d₆): δ 3.11 (br s, 4H), 3.17 (br s, 4H), 6.58 (d, J=16.0 Hz, 1H), 6.66 (dd, J=8.0, 2.2 Hz, 1H), 6.90 (s, 1H), 6.95 (d, J=7.8 Hz, 1H), 7.14 (t, J=7.8 Hz, 1H), 7.29 (d, J=16.0 Hz, 1H), 7.44 (s, Hz, 1H), 7.72 (d, J=8.9 Hz, 2H), 7.93 (d, J=8.9 Hz, 2H), 8.99 (br s, 2H), 11.15 (br s, 1H). MS (ES+): m/z 443 (M+H)⁺.

### Example 31

### 4-(6-Chloro-2-methyl-pyrimidin-4-yl)-piperazine-1-carboxylic acid tert-butyl ester (Intermediate 19)

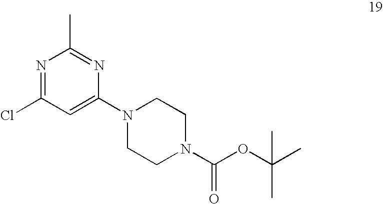

4,6-Dichloro-2-methyl-pyrimidine (5.8 g, 35.6 mmol) and piperazine-1-carboxylic acid tert-butyl ester (6.6 g, 35.6 mmol) were ground together in mortar and pestle. During the grinding process, solids gradually form waxy paste then white powder once reaction is complete. Solids were removed, diluted with EtOAc and washed with saturated sodium bicarbonate solution. Organic phase was dried and evaporated to white solids. Trituration with hexanes provided title intermediate (3.3 g, 30%).

¹H NMR (500 MHz, CDCl₃): δ 1.48 (s, 9H), 2.48 (s, 3H), 3.51 (t, J=4.8 Hz, 4H), 3.63 (m, 4H), 6.32 (s, 1H). MS (ES+): m/z 213 (M-Boc+H)⁺.

### Example 32

### 4-[6-(5-Formyl-thiazol-2-ylamino)-2-methyl-pyrimidin-4-yl]-piperazine-1-carboxylic acid tert-butyl ester (Intermediate 20)

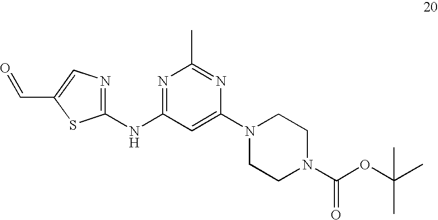

In a dry 100 mL round bottom flask were combined intermediate 19 (2.8 g, 8.9 mmol), 2-amino-thiazole-5-carbaldehyde (1.4 g, 10.7 mmol), cesium carbonate (8.7 g, 26.7 mmol), Xantphos (1.03 g, 1.8 mmol) and tris(dibenzylideneacetone) dipalladium (0.82 g, 0.89 mmol). Reactants were diluted with dioxane (40 mL), flushed with argon and refluxed for 7 h. Reaction was then diluted with EtOAc and filtered. Solvents were then removed and resulting residue purified via column chromatography (silica gel) to provide title intermediate as yellow powder (1.5 g, 42%).

¹H NMR (500 MHz, DMSO-d₆): δ 1.42 (s, 9H), 2.42 (s, 3H), 3.42 (m, 4H), 3.54 (m, 4H), 6.10 (s, 1H), 8.30 (s, 1H), 9.85 (s, 1H), 11.90 (s, 1H). MS (ES+): m/z 405 (M+H)⁺.

### Example 33

### 4-[2-Methyl-6-(5-vinyl-thiazol-2-ylamino)-pyrimidin-4-yl]-piperazine-1-carboxylic acid tert-butyl ester (Intermediate 21)

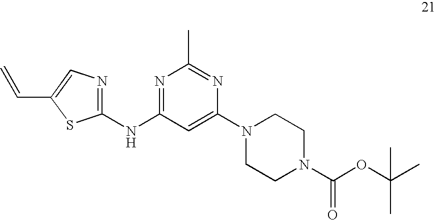

A stirring solution of methyl triphenyl phosphonim bromide (1.8 g, 5 mmol) in 20 mL THF was treated with 2.5M butyllithium solution in hexanes (2.3 mL, 5.9 mmol). Additional 0.6 mL BuLi was added until clear orange solution resulted. After 20 min, intermediate 20 (0.82 g, 2 mmol) was added as a solid in one portion. After 16h, reaction solvents were evaporated to dark residue which was purified via column chromatography (silica gel) to afford title intermediate as brown solid (0.6 g, 74%).

### Example 34

### N-(5-((E)-2-(7-Chloro-1H-indol-4-yl)vinyl)thiazol-2-yl)-2-methyl-6-(piperazin-1-yl)-pyrimidin-4-amine (Compound XII)

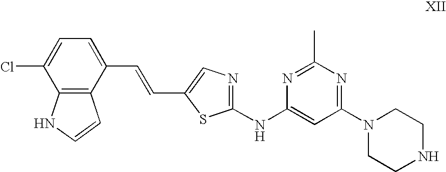

To a solution of intermediate 21 (402.5 mg, 1.0 mmol) in DMF (10 mL) was added 4-bromo-7-chloro-1H-indole (254 mg, 1.1 mmol), Pd(OAc)₂ (45 mg, 0.2 mmol), and Et₃N (0.56 mL, 4.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) added. The reaction was stirred for 4 h at room temperature. 10% NaOH solution (20 mL) was added. The organic layer was separated and aqueous extracted with CH₂Cl₂ (2×10 mL). The combined organic layer was dried (Na₂SO₄) and the solvent removed in vacuo. The residue was purified by HPLC to afford the title compound (15 mg of HCl salt, 3%) as a yellow solid.

¹H NMR (500 MHz, DMSO-d₆) δ 2.51 (s, 3H), 3.18 (br s, 4H), 3.79 (br s, 4H), 6.92 (dd, J=3.0, 1.9 Hz, 1H), 7.05 (d, J=16.4 Hz, 1H), 7.15 (d, J=8.0 Hz, 1H), 7.25 (d, J=8.0 Hz, 1H), 7.47 (t, J=2.8 Hz, 1H), 7.48 (d, J=16.4 Hz, 1H), 9.31 (br s, 2H), 11.53 (br s, 1H). MS (ES+): m/z 452 (M+H)⁺.

### Example 35

### 4-Hydroxy-piperdine-1-carboxylic acid tert-butyl ester (Intermediate 22)

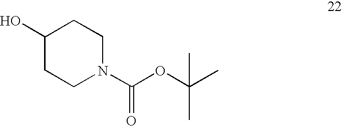

To a solution of piperidin-4-ol (153 g, 1.51 mol) and Et₃N (210 g, 2.08 mol) in MeOH (800 mL) was added dropwise a solution of di-tert-butyl dicarbonate (350 g, 1.60 mol) in MeOH (200 mL) under ice cooling. After the addition was complete, the resulting mixture was stirred at room temperature for 24 h. Upon completion, the reaction mixture was concentrated, and the residue was partitioned between 1N aqueous HCl solution (1000 mL) and EtOAc. The organic layer was dried over MgSO₄, and concentrated to give the title intermediate (275 g, 90%).

### Example 36

### 4-Methanesulfonyloxy-piperidine-1-carboxylic acid tert-butyl ester (Intermediate 23)

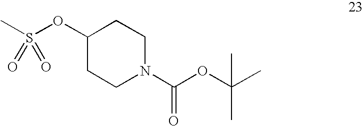

To a solution of intermediate 22 (200 g, 1.0 mol) and Et₃N (204 g, 2.0 mol) in CH₂Cl₂ (3000 mL) was added dropwise a solution of MsCl (130 g, 1.14 mol) in CH₂Cl₂ (500 mL) under ice cooling. After the addition completed, the resulting mixture was stirred at room temperature for 24 h. Upon completion, the reaction mixture was washed with water and 1N aqueous HCl (1000 mL). The organic layer was dried over MgSO₄, and concentrated to give the title intermediate (230 g, 82%) as a white solid.

### Example 37

### 4-(4-Bromo-phenylsulfanyl)-piperidine-1-carboxylic acid tert-butyl ester (Intermediate 24)

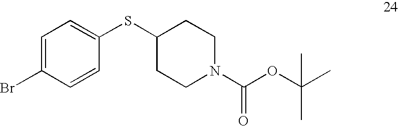

Intermediate 23 (82.0 g, 0.29 mol), 4-bromothiophenol (50.0 g, 0.27 mol) and K₂CO₃ (80.0 g, 0.58 mol) were mixed in CH₃CN (5000 mL) at room temperature. The mixture was heated to reflux and stirred for 24 h. Upon completion, the reaction mixture was filtered and the filtrate was concentrated. The residue was dissolved in EtOAc and washed with 1N NaOH. The organic layer was concentrated to give crude product, which was purified by column chromatography (silica, elute; petroleum: EtOAc=10:0, 10:1) to give the title intermediate (62 g, 79%) as a white solid.

### Example 38

### 4-(4-Bromo-benzenesulfonyl)-piperidine-1-carboxylic acid tert-butyl ester (Intermediate 25)

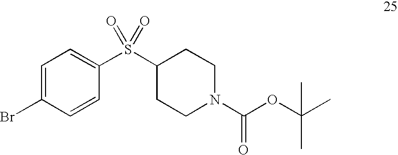

Water (40 mL) was added to alumina (140 g, 1.37 mol) at room temperature and stirred for 5 min. To the resulting slurry was added CHCl₃ (800 mL), a solution of intermediate 24 (62 g, 0.17 mol) in CHCl₃ (900 mL) and oxone (170 g, 0.28 mol) successively. The resulting slurry was heated to reflux and stirred for 24 h. Upon completion, the reaction mixture was cooled to room temperature and then filtered. The filtrate was washed with saturated aqueous Na₂SO₃ solution, dried over MgSO₄, and evaporated to give crude product, which was purified by re-crystallization from petroleum/EtOAc (2000 ml/500 ml) to give the title intermediate (59 g, 87%) as a white solid.

### Example 39

### 4-[4-(5-Formyl-thiazol-2-ylamino)-benzenesulfonyl]-piperidine-1-carboxylic acid tert-butyl ester (Intermediate 26)

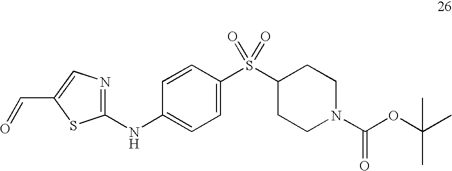

In a dry 15 mL microwave vial were combined 2-amino-thiazole-5-carbaldehyde (0.354 g, 2.77 mmol), intermediate 25 (1.23 g, 3.04 mmol), cesium carbonate (2.7 g, 8.29 mmol), Xantphos (0.32 g, 0.55 mmol) and tris(dibenzylideneacetone) dipalladium (0.25 g, 0.28 mmol). Reactants were diluted with dioxane (12 mL), flushed with argon and irradiated for 15 min at 160° C. Reaction was then diluted with EtOAc, decanted and washed with water followed by brine. Organic phase cut from aqueous layer, dried over sodium sulfate, filtered and evaporated to dark oil. Crude product was purified via silica gel chromatography. The title intermediate was isolated as pale yellow solids (0.34 g, 27%).

### Example 40

### 4-(4-{5-[2-(4-Nitro-phenyl)-vinyl]-thiazol-2-ylamino}-benzenesulfonyl)-piperidine-1-carboxylic acid tert-butyl ester (Intermediate 27)

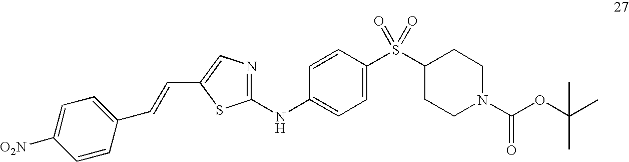

(4-Nitro-benzyl)-triphenyl-phosphonium bromide (0.2 g, 0.42 mmol) was dissolved in 150 mL DCM and washed with 1N NaOH. Resulting bright red solution was then dried over sodium sulfate, filtered and poured into 500 mL round bottom flask. Approximately 60 mL anhydrous toluene was added and solvents were reduced under rotary evaporation until only 40-50 mL of solvent remained. This was then added to a stirring premade solution of intermediate 26 (0.15 g, 0.33 mmol) and benzoic acid (0.01 g) in toluene (25 mL). Reaction immediately heated to 90° C. and stirred at that temperature overnight. After 16 h, reaction was cooled to room temperature, solvents were removed and residue used without further purification. (0.2 g, 100%).

### Example 41

### {5-[2-(4-Nitro-phenyl)-vinyl]-thiazol-2-yl}-[4-(piperidine-4-sulfonyl)-phenyl]-amine (Compound XIII)

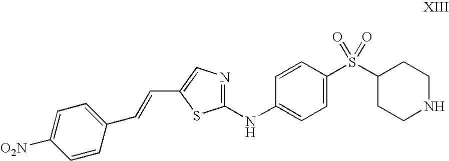

A stirring solution of intermediate 27 (0.022 g, 0.039 mmol) in DCM (5 mL) was treated with TFA (0.2 mL) and allowed to stir at room temperature for 2 h. Solvents were then removed and residue purified via HPLC. Title compound isolated as orange solids (0.013 g, 72%). ¹H NMR (DMSO-d₆): δ 1.66 (qd, J=13.0 Hz, 3.8 Hz, 2H), 2.02 (d, J=12.9 Hz, 2H), 2.84-2.88 (m, 2H), 3.49 (tt, J=12.0, 3.4 Hz, 1H), 6.83 (d, J=16.0 Hz, 1H), 7.58 (s, 1H), 7.71 (d, J=16 Hz, 1H), 7.78-7.81 (m, 4H), 7.93 (d, J=8.9 Hz, 2H), 8.21 (d, J=8.9 Hz, 2H), 8.63 (br s, 1H), 11.2 (s, 1H). MS (ES+): m/z 471 (M+H)⁺.

### Example 42

### 4-(3-{5-[2-(4-Methoxy-phenyl)-vinyl]-thiazol-2-ylamino}-phenyl)-piperazine-1-carboxylic acid tert-butyl ester (Intermediate 28)

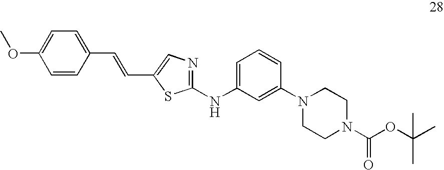

In a dry 15 mL microwave vial were combined intermediate 3 (0.09 g, 0.3 mmol), intermediate 17 (0.93 g, 0.34 mmol), cesium carbonate (0.3 g, 0.92 mmol), Xantphos (0.035 g, 0.06 mmol) and tris(dibenzylideneacetone) dipalladium (0.028 g, 0.034 mmol). Reactants were diluted with dioxane (12 mL), flushed with argon and irradiated for 15 min at 160° C. Reaction was then diluted with EtOAc, decanted and washed with water followed by brine. Organic phase cut from aqueous layer, dried over sodium sulfate, filtered and evaporated to orange solids that were used without further purification (0.097 g, 65%).

### Example 43

### 4-{2-[2-(3-Piperazin-1-yl-phenylamino)-thiazol-5-yl]-vinyl}-phenol (Compound XIV)

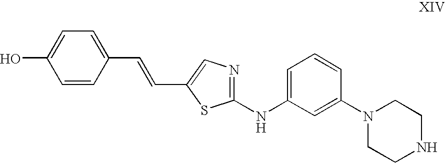

Intermediate 28, (0.077 g, 0.16 mmol) was diluted with 10 mL DCM and chilled to 0° C. A 1.0 M solution of BBr₃ in DCM (0.8 mL, 0.8 mmol) was then added in several portions resulting in dark reaction mixture. Once addition was complete, reaction was allowed to come to ambient temperature and stir for 1 h. Reaction was then quenched by carefully adding methanol (ca. 5 mL) and then evaporating solvents. Resulting residue purified by HPLC to provide title compound as yellow powder (0.005 g, 8% yield).

¹H NMR (DMSO-d₆): δ 3.26 (br s, 4H), 3.32 (br s, 4H), 6.49 (d, J=16.0 Hz, 1H), 6.63 (dd, J=8.1, 2.0 Hz, 1H), 6.73 (d, J=8.7 Hz, 2H), 7.05 (d, J=9.5 Hz, 1H), 7.09 (d, J=16.0 Hz, 1H), 7.17 (t, J=8.1 Hz, 1H), 7.20 (s, 1H), 7.23-7.35 (m, 3H), 7.95 (s, 2H), 8.73 (br s, 1H), 9.56 (br s, 1H), 10.24 (br s, 1H). MS (ES+): m/z 379 (M+H)⁺.

### Example 44

### 4-(6-{5-[2-(1H-Indol-5-yl)-vinyl]-thiazol-2-ylaminol}-2-methyl-pyrimidin-4-yl)-piperazine-1-carboxylic acid tert-butyl ester (Intermediate 29)

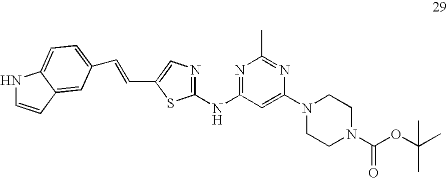

A stirring solution of intermediate 8 (0.39 g, 0.68 mmol) in 25 mL THF was treated with 2.5M butyllithium solution in hexanes (0.33 mL, 0.82 mmol). A turbid, yellow slurry persisted. Additional butyllithium (0.6 mL) was added so as to result in a homogeneous red solution. After 20 min, intermediate 20 (0.11 g, 0.27 mmol) was added as a solid in one portion. After 3 h, the reaction was poured onto water and extracted with DCM. Organic phase was then washed with brine, dried over sodium sulfate, filtered and evaporated to dark residue. Column chromatography (silica gel) afforded title intermediate as pale yellow solids (0.06 g, 43%).

### Example 45

### {5-[2-(1H-Indol-5-yl)-vinyl]-thiazol-2-yl}-(2-methyl-6-piperazin-1-yl-pyrimidin-4-yl)-amine (Compound XV)

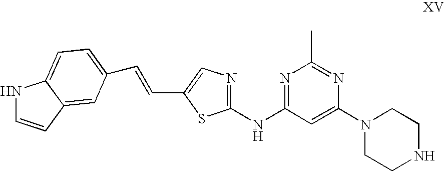

A stirring solution of intermediate 29 (0.05 g, 0.097 mmol) in DCM (5 mL) was treated with TFA (0.2 mL) and allowed to stir at room temperature for 18 h. Solvents were then removed and residue purified via HPLC. Title compound isolated as white solids (0.006 g, 15%). ¹H NMR (DMSO-d₆): δ 1.85 (s, 3H), 3.15 (br s, 4H), 3.19 (br s, 4H), 5.98 (s, 1H), 6.42 (s, 1H), 6.60-6.68 (m, 2H), 7.03 (dd, J=6.2, 2.0 Hz, 1H), 7.33-7.52 (m, 6H), 8.78 (br s, 2H), 11.06 (br s, 1H), 11.12 (br s, 1H). MS (ES+): m/z 418 (M+H)⁺.

### Example 46

### (4-Bromo-indazol-1-yl)-phenyl-methanone (Intermediate 30)

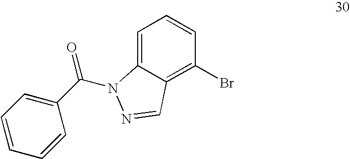

A stirring solution of 4-bromo-1H-indazole (1.01 g, 5.13 mmol) was treated with TEA (1.7 mL, 12.3 mmol) and chilled to 0° C. Benzoyl chloride (0.84 mL, 7.2 mmol) was then added slowly as a solution in 5 mL DCM. Reaction was allowed to come to room temperature and stir for 3 h. Solvents were then poured onto water and treated with saturated sodium bicarbonate solution. Organic phase was dried over sodium sulfate, filtered and evaporated to brown residue. Trituration with small amount of methanol afforded title intermediate as beige solids (1.3 g, 85%).

### Example 47

### {5-[2-(1H-Indazol-4-yl)-vinyl]-thiazol-2-yl}-(2-methyl-6-piperazin-1-yl-pyrimidin-4-yl)-amine (Compound XVI)

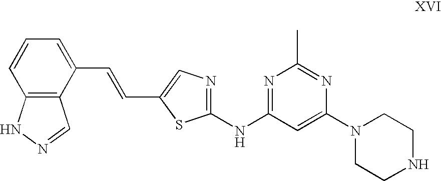

In a dry 15 mL microwave vial were combined intermediate 30 (0.18 g, 0.6 mmol), intermediate 21 (0.2 g, 0.49 mmol), cesium carbonate (0.32 g, 1 mmol), Xantphos (0.058 g, 0.1 mmol) and tris(dibenzylideneacetone) dipalladium (0.046 g, 0.05 mmol). Reactants were diluted with dioxane (12 mL), flushed with argon and irradiated for 15 min at 160° C. Reaction was then diluted with EtOAc, decanted and washed with water followed by brine. Organic phase cut from aqueous layer, dried over sodium sulfate, filtered and evaporated to dark residue that was diluted with DCM (25 mL) and treated with 20% TFA in DCM solution. Solvents were immediately evaporated and resulting residue was purified via HPLC to afford title intermediate as white solids (0.001 g, 0.5%).

¹H NMR (DMSO-d₆): δ 3.2 (br s, 4H), 3.73 (br s, 4H), 6.16 (s, 1H), 7.08 (d, J=16.2 Hz, 1H), 7.27-7.34 (m, 2H), 7.43 (d, J=8.0 Hz, 1H), 7.60 (s, 1H), 7.64 (d, J=16.0 Hz, 1H), 8.53 (br s, 1H), 8.81 (br s, 2H). MS (ES+): m/z 419 (M+H)⁺.

### Example 48

### 4-(6-Chloro-pyrimidin-4-yl)-piperazine-1-carboxylic acid tert-butyl ester (Intermediate 31)

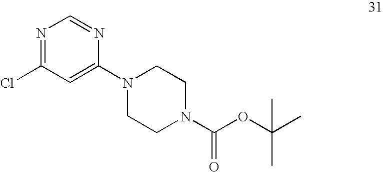

A stirring solution of 4,6-dichloro-pyrimidine (2.2 g, 15 mmol) in dioxane (50 mL) was treated with TEA (6.2 mL, 45 mmol) and piperazine-1-carboxylic acid tert-butyl ester (3.5 g, 18.8 mmol). This was then heated to 75° C. and stirred for 4 h. Solvents then cooled to room temperature and evaporated. Crude product purified by column chromatography (silica gel) to afford title intermediate as white powder (2.9 g, 64%).

### Example 49

### 4-[6-(5-Formyl-thiazol-2-ylamino)-pyrimidin-4-yl]-piperazine-1-carboxylic acid tert-butyl ester (Intermediate 32)

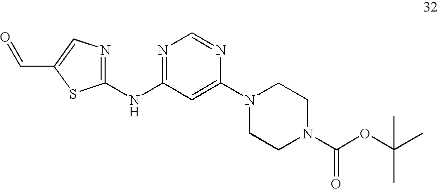

In a dry 15 mL microwave vial were combined intermediate 31 (1.1 g, 3.7 mmol), 2-amino-thiazole-5-carbaldehyde (0.56 g, 4.4 mmol), cesium carbonate (3.6 g, 11 mmol), Xantphos (0.42 g, 0.73 mmol) and tris(dibenzylideneacetone) dipalladium (0.34 g, 0.37 mmol). Reactants were diluted with dioxane (12 mL), flushed with argon and irradiated for 15 min at 160° C. Reaction was then diluted with EtOAc and filtered. Solvents were then removed and resulting residue purified via column chromatography (silica) to provide title intermediate as brown powder (1 g, 70%).

### Example 50

### (3-Methoxy-benzyl)-triphenyl-phosphonium bromide (Intermediate 33)

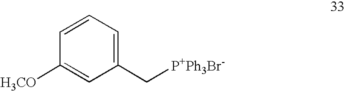

A stirring solution of 1bromomethyl-3-methoxy-benzene (10 g, 50 mmol) in toluene (150 mL) was treated with triphenyl phosphine (15 g, 57 mmol) and heated to reflux. After 6 h, reaction was cooled to room temperature and title intermediate was filtered off, washed with cold toluene dried (22 g, 97%).

¹H NMR (500 MHz, DMSO-d₆): 3.49 (s, 3H), 6.46 (m, 1H), 6.59 (d, J=7.7 Hz, 1H), 6.86 (dt, J=8.2, 2.3 Hz, 1H), 7.16 (t, J=7.7 Hz, 1H), 7.32-7.36 (m, 1H), 7.64-7.68 (m, 6H), 7.73-7.75 (m, 6H), 7.91 (m, 3H). MS (ES+): m/z 383 (M-Br+H)⁺.

### Example 51

### 4-(6-{5-[2-(3-Methoxy-phenyl)-vinyl]-thiazol-2-ylaminol}-pyrimidin-4-yl)-piperazine-1-carboxylic acid tert-butyl ester (Intermediate 34)

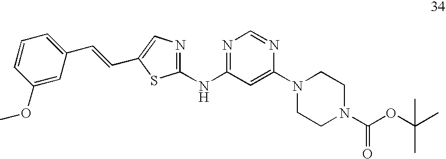

A stirring solution of intermediate 33 (1 g, 2.2 mmol) in 25 mL THF was treated with 2.5M butyllithium solution in hexanes (1 mL, 2.5 mmol). After 20 min, intermediate 32 (0.48 g, 1.2 mmol) was added as a solid in one portion. After 16 h, reaction solvents were evaporated to dark residue. Column chromatography (silica gel) afforded title intermediate as off white powder (0.35 g, 57%).

### Example 52

### 3-{2-[2-(6-Piperazin-1-yl-pyrimidin-4-ylamino)-thiazol-5-yl]-vinyl}-phenol (Compound XVII)

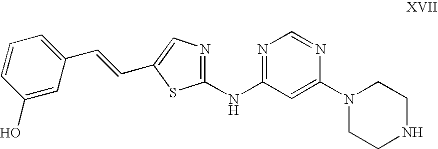

Intermediate 34, (0.27 g, 0.55 mmol) was diluted with 20 mL DCM and chilled to 0° C. A 1.0 M solution of BBr₃ in DCM (3.8 mL, 3.8 mmol) was then added in several portions resulting in dark reaction mixture. Once addition was complete, reaction was allowed to come to ambient temperature and stir for 2 h. Reaction was then quenched by carefully adding methanol (ca. 10 mL) and then evaporating solvents. Resulting residue purified by HPLC to provide title compound as yellow powder (0.053 g, 26% yield).

¹H NMR (DMSO-d₆): δ 2.74-2.76 (m, 4H), 3.43-3.45 (m, 4H), 6.18 (br s, 1H), 6.63-6.66 (m, 2H), 6.89 (t, J=1.9 Hz, 1H), 6.95 (d, J=7.9 Hz, 1H), 7.13 (t, J=7.8 Hz, 1H), 7.26 (d, J=16.0 Hz, 1H), 7.43 (s, 1H), 8.35 (s, 1H). MS (ES+): m/z 381 (M+H)⁺.

### Example 53

### 4-(6-Chloro-2-methyl-pyrimidin-4-yl)-[1,4]diazepane-1-carboxylic acid tert-butyl ester (Intermediate 35)

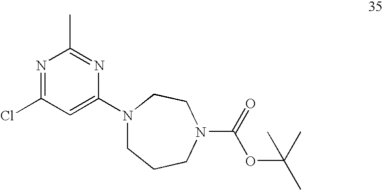

A stirring solution of 4,6-dichloro-2-methyl-pyrimidine (1.7 g, 11.4 mmol) in dioxane (50 mL) was treated with TEA (4.7 mL, 34 mmol) and [1,4]diazepane-1-carboxylic acid tert-butyl ester (2.7 mL, 14 mmol). This was then heated to 75° C. and stirred for 4 h. Solvents then cooled to room temperature and poured onto water resulting in white precipitate. Title intermediate filtered off and dried (2 g, 54%).

### Example 54

### 4-[6-(5-Formyl-thiazol-2-ylamino)-2-methyl-pyrimidin-4-yl]-[1,4]diazepane-1-carboxylic acid tert-butyl ester (Intermediate 36)

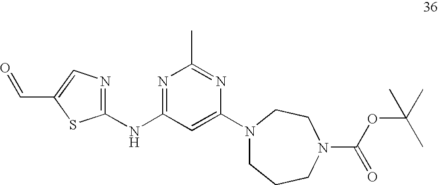

In a dry 15 mL microwave vial were combined intermediate 35 (0.35 g, 1 mmol), 2-amino-thiazole-5-carbaldehyde (0.12 g, 0.9 mmol), cesium carbonate (0.58 g, 1.8 mmol), Xantphos (0.052 g, 0.09 mmol) and tris(dibenzylideneacetone) dipalladium (0.041 g, 0.045 mmol). Reactants were diluted with dioxane (12 mL), flushed with argon and irradiated for 15 min at 160° C. Reaction was then diluted with EtOAc and filtered. Solvents were then removed and resulting residue purified via column chromatography (silica gel) to provide title intermediate as brown powder (0.11 g, 29%).

### Example 55

### 4-(6-{5-[2-(3-Methoxy-phenyl)-vinyl]-thiazol-2-ylaminol}-2-methyl-pyrimidin-4-yl)-[1,4]diazepane-1-carboxylic acid tert-butyl ester (Intermediate 37)

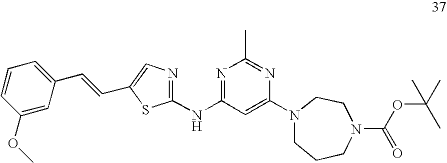

A stirring solution of intermediate 33 (0.1 g, 0.22 mmol) in 5 mL THF was treated with 2.5M butyllithium solution in hexanes (0.12 mL, 0.3 mmol). After 20 min, 36 (0.062 g, 0.15 mmol) was added as a solid in one portion. After 16 h, reaction solvents were evaporated to dark residue which was used without further purification. (0.06 g, 75%).

### Example 56

### 3-{2-[2-(6-[1,4]Diazepan-1-yl-2-methyl-pyrimidin-4-ylamino)-thiazol-5-yl]-vinyl}-phenol (Compound XVIII)

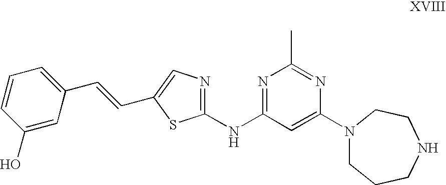

Intermediate 37 (0.05 g, 0.1 mmol) was diluted with 10 mL DCM and chilled to 0° C. A 1.0 M solution of BBr₃ in DCM (1 mL, 1 mmol) was then added in several portions resulting in dark reaction mixture. Once addition was complete, reaction was allowed to come to ambient temperature and stir for 1 h. Reaction was then quenched by carefully adding methanol (ca. 2 mL) and then evaporating solvents. Resulting residue purified by HPLC to provide title compound as white powder (0.007 g, 18% yield).

¹H NMR (DMSO-d₆): δ 2.06 (br s, 2H), 2.46 (s, 3H), 3.18 (br s, 2H), 3.28 (br s, 2H), 3.59 (br s, 2H), 6.05 (s, 1H), 6.63-6.66 (m, 2H), 6.89 (s, 1H), 6.96 (d, J=7.9 Hz, 1H), 7.14 (d, J=7.8 Hz, 1H), 7.27 (d, J=16.0 Hz, 1H), 7.45 (s, 1H), 8.69 (br s, 2H), 9.4 (br s, 1H), 11.23 (br s, 1H). MS (ES+): m/z 409 (M+H)⁺.

### Example 57

### 6-[(E)-2-{2-[(2-Methyl-6-piperazin-1-ylpyrimidin-4-yl)amino]-1,3-thiazol-5-yl}vinyl]-1,3-benzothiazol-2-amine (Compound XIX)

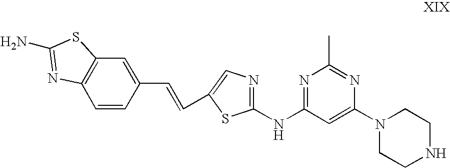

5 mL Emrys microwave vial was charged with intermediate 21 (80.5 mg, 0.2 mmol), 2-amino-6-bromobenzothiazole (138.0 mg, 0.6 mmol), Pd₂(dba)₃ (36.6 mg, 0.004 mmol), tri(tert-butyl)phosphine (0.16 mL of 1.0 M solution in toluene, 0.16 mmol), cesium carbonate 130.3 mg, 0.4 mmol) and anhydrous dioxane (5 mL). The reaction mixture was purged with argon gas for 10 min, then sealed and irradiated in a microwave (Initiator, Biotage) at 180° C. for 60 min. After cooling to ambient temperature, the reaction mixture was diluted with ca. 50 mL of EtOAc and filtered through a short pad of silica gel. The silica gel pad was washed with 10% MeOH in EtOAc. Combined organic solutions were concentrated in vacuo with ca. 5 g of silica gel. The loaded silica gel was taken to the ISCO system for further purification (4 g RediSep column Teledyne ISCO, solid method, 0% to 100% EtOAc gradient in hexanes, 15 min method). Fractions, containing the product, were combined and concentrated in vacuo to give a yellow solid. This solid was treated with 30% TFA in DCM for 10 min and the resulting solution was concentrated in vacuo to give yellow oil. The oil was re-dissolved in 3 mL of DMF, filtered through 0.2 micron syringe filter and purified by reverse-phase preparative HPLC in CH₃CN/H₂O system containing 0.05% of TFA. All fractions, containing the product, were combined and solvent was removed in vacuo to give the TFA salt of the title compound as a bright-yellow solid (12.8 mg, 11%).

¹H NMR (500 MHz, DMSO-d₆): δ 2.46 (s, 3H), 3.16-3.20 (m, 4H), 3.72-3.75 (m, 4H), 6.15 (s, 1H), 6.77 (d, J=16.1 Hz, 1H), 7.30 (d, J=16.2 Hz, 1H), 7.34 (d, J=8.4 Hz, 1H), 7.42 (s, 1H), 7.48 (dd, J=8.5, 1.6 Hz, 1H), 7.92 (d, J=1.4 Hz, 1H), 8.26 (br s, 2H), 8.87 (s, 2H), 11.36 (br s, 1H). MS (ES+): m/z 451 (M+H)⁺.

### Example 58

### (E)-tert-Butyl 4-(6-(5-(3-methoxystyryl)thiazol-2-ylamino)-2-methylpyrimidin-4-yl)piperazine-1-carboxylate (Intermediate 38)

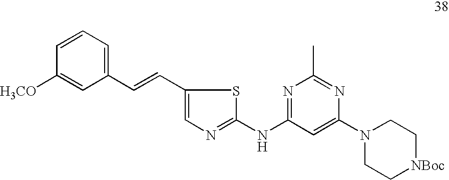

To a solution of intermediate 33 (6.0 g, 13.0 mmol) in THF (80 mL) at 0° C. was added a solution of 2.5 M n-butyl lithium in hexanes (8.3 mL, 20.8 mmol). The reaction mixture was stirred 30 min at 0° C. followed by addition of intermediate 20 (2.1 g, 5.2 mmol). The reaction mixture was stirred for 60 min at room temperature and then purified by silica gel chromatography (DCM:EtOAc 100:0 to 95:5 gradient) to afford the title intermediate as a brown solid (1.36 g, 52%). MS (ES+): m/z 509 (M+H)⁺.

### Example 59

### (E)-3-(2-(2-(2-Methyl-6-(piperazin-1-yl)pyrimidin-4-ylamino)thiazol-5-yl)vinyl)phenol hydrochloride (Compound XX)

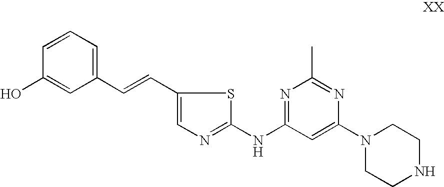

To a solution of intermediate 38 (1.00 g, 1.97 mmol) in DCM (25 mL) at 0° C. was added a solution of 1 M BBr₃ in DCM (9.8 mL, 9.8 mmol) dropwise. The reaction mixture was stirred for 30 min at room temperature and purified by HPLC. Fractions that contained the title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (327 mg, 42%).

¹H NMR (500 MHz, DMSO-d₆): δ 2.46 (s, 3H), 3.16 (m 4H), 3.77 (m, 4H), 6.18 (s, 1H), 6.65 (m, 1H), 6.65 (d, J=15.8 Hz, 1H), 6.91 (s, 1H), 6.95 (d, J=7.8 Hz, 1H), 7.13 (t, J=7.9 Hz, 1H), 7.27 (d, J=16.2 Hz, 1H), 7.45 (s, 1H), 9.53 (br s, 3H), 11.4 (br s, 1H). MS (ES+): m/z 395 (M+H)⁺.

### Example 60

### (E)-5-(3-Methoxystyryl)-N-(2-methyl-6-(piperazin-1-yl)pyrimidin-4-yl)thiazol-2-amine hydrochloride (Compound XXI)

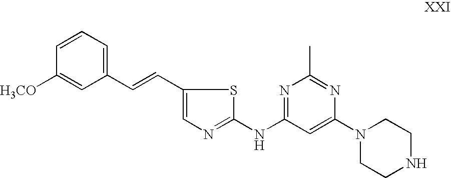

To a solution of intermediate 38 (0.30 g, 0.59 mmol) in DCM (10 mL) was added trifluoroacetic acid (1 mL). The reaction mixture was stirred overnight at room temperature and purified by HPLC. Fractions that contained the title compound were combined and neutralized with aqueous NaHCO₃ and extracted with EtOAc. The organic layer was treated with 4 M HCl in 1,4-dioxane (1 mL) and concentrated in vacuo to afford the title compound as a yellow solid (277 mg, quant).

¹H NMR (500 MHz, DMSO-d₆): δ 2.47 (s, 3H), 3.17 (m, 4H), 3.77 (s, 3H), 6.15 (s, 1H), 6.20 (s, 1H), 6.74 (d, J=16.2 Hz, 1H), 6.90 (dd, J=7.8, 2.3 Hz, 1H), 7.11 (s, 1H), 7.12 (s, 1H), 7.25 (t, J=8.1 Hz, 1H), 7.40 (t, J=16.2 Hz, 1H), 7.46 (s, 1H), 9.40 (br s, 3H), 10.70-11.70 (br s, 1H). MS (ES+): m/z 409 (M+H)⁺.

### Example 61

### {5-[2-(4-Methoxy-phenyl)-vinyl]-thiazol-2-yl}-[3-(2-pyrrolidin-1-yl-ethoxy)-phenyl]-amine (Intermediate 39)

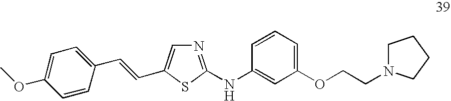

A suspension of intermediate 3 (0.14 g, 0.47 mmol), 3-(2-pyrrolidin-1-yl-ethoxy)-phenylamine (0.13 g, 0.63 mmol), Pd₂(dba)₃ (25 mg, 0.027 mmol), Xantphos (32 mg, 0.055 mmol) and cesium carbonate (0.3 g, 0.92 mmol) in dioxane (20 mL) was heated at reflux under the argon atmosphere for 21 h. After cooling to room temperature, the mixture was filtered and the filtered solid washed with DCM. The filtrate was concentrated and the residue purified by flash chromatography on silica gel (10% MeOH/DCM to 30% MeOH/DCM) to afford the title intermediate as a light brown solid (70 mg, 35%).

### Example 62

### 4-(2-{2-[3-(2-Pyrrolidin-1-yl-ethoxy)-phenylamino]-thiazol-5-yl}-vinyl)-phenol (Compound XXII)

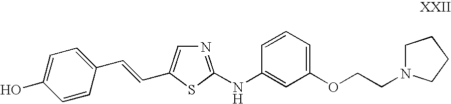

To a suspension of intermediate 39 (70 mg, 0.17 mmol) in DCM (10 mL) at 0° C. was added BBr₃ (1 M in DCM; 0.5 mL, 0.5 mmol) and the mixture stirred at room temperature for 2 h. The reaction was quenched with saturated NaHCO₃ solution until the pH ˜7. The resulting solution was extracted with EtOAc (30 mL) and the extract washed with brine, dried over anhydrous Na₂SO₄ and filtered. The filtrate was concentrated and the residue purified by HPLC. The fractions were combined and poured into saturated NaHCO₃ solution (30 mL). The combined aqueous layers were extracted with EtOAc (2×30 mL) and the combined organic layers washed with brine, dried over anhydrous Na₂SO₄ and filtered. The filtrate was concentrated and the residue re-dissolved in MeOH (5 mL). HCl (2 M in Et₂O, 0.1 mL) was added and the mixture stirred at RT for 3 min. The reaction mixture was concentrated to afford the title compound as a brown solid (HCl salt; 3 mg, 4%).

¹H NMR (500 MHz, DMSO-d₆): δ 1.85-1.95 (m, 2H), 1.95-2.05 (m, 2H), 3.05-3.15 (m, 2H), 3.50-3.60 (m, 4H), 4.32 (t, J=4.8 Hz, 2H), 6.50 (d, J=16.1 Hz, 1H), 6.61 (dd, J=8.0, 2.3 Hz, 1H), 6.74 (d, J=8.5 Hz, 2H), 7.11 (d, J=15.7 Hz, 1H), 7.10-7.15 (m, 1H), 7.24 (t, J=8.1 Hz, 1H), 7.26 (s, 1H), 7.33 (d, J=8.6 Hz, 2H), 7.51 (s, 1H), 9.58 (br s, 1H), 10.46 (s, 1H), 10.50 (br s, 1H). MS (ES+): m/z 408 (M+H)⁺.

### Example 63

### 2-Bromo-5-[2-(4-nitro-phenyl)-vinyl]-thiazole (Intermediate 40)

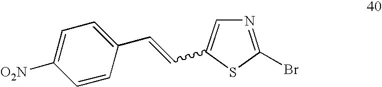

To a solution of (4-nitro-benzyl)-triphenyl-phosphonium bromide (1.6 g, 3.4 mmol) in anhydrous DMF (20 mL) was added BuOK (0.40 g, 3.6 mmol). The solution was stirred for 30 min before adding solution of 2-bromo-thiazole-5-carbaldehyde (0.50 g, 2.6 mmol) in DMF (10 mL). The mixture was stirred overnight and poured into water. The mixture was extracted with EtOAc (2×40 mL) and the combined organic extracts washed with brine, dried over anhydrous Na₂SO₄ and filtered. The filtrate was concentrated and the residue purified by flash column on silica gel (hexanes to 15% EtOAc/hexanes) to afford title intermediate as a mixture of cis/trans isomers (0.25 g, 31%).

### Example 64

### 4-[2-(2-Bromo-thiazol-5-yl)-vinyl]-phenylamine (Intermediate 41)

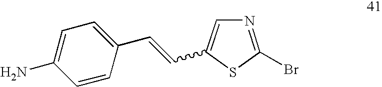

A mixture of intermediate 40 (0.40 g, 1.3 mmol) and sodium sulfide (1.0 g, 4.2 mmol) in ethanol (15 mL) was heated at reflux for 1 h. After cooling to RT, the mixture was poured into water. The aqueous layer was extracted with EtOAc and the organic layer separated. The organic extract was washed with brine, dried over anhydrous Na₂SO₄ and then filtered. The filtrate was concentrated and used in the next step without purification.

### Example 65

### N-{4-[2-(2-Bromo-thiazol-5-yl)-vinyl]-phenyl}-3-trifluoromethyl-benzamide (Intermediate 42)

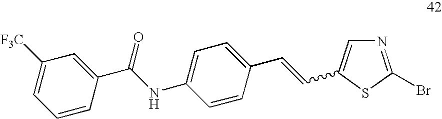

To a solution of intermediate 41 (0.28 g, 1.0 mmol) in CH₂Cl₂ (15 mL) was added 3-trifluoromethyl-benzoyl chloride (0.25 mL, 1.7 mmol) and Et₃N (0.50 mL, 3.6 mmol). The reaction mixture was stirred at RT for 1.5 h. Saturated NaHCO₃ (30 mL) was added and the mixture extracted with EtOAc (40 mL). The organic layer was separated and washed with brine, dried over anhydrous Na₂SO₄ and filtered. The filtrate was concentrated and the residue purified by column chromatography on silica gel (hexanes to 30% EtOAc/hexanes) to afford the title intermediate as a yellow solid (0.35 g, 77%).

### Example 66

### N-(4-{2-[2-(3-Piperazin-1-yl-phenylamino)-thiazol-5-yl]-vinyl}-phenyl)-3-trifluoromethyl-benzamide (Compound XXIII)

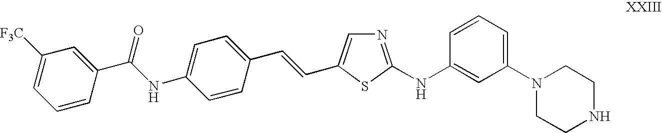

A suspension of intermediate 42 (0.15 g, 0.33 mmol), intermediate 17 (0.12 g, 0.43 mmol), Pd₂(dba)₃ (20 mg, 0.022 mmol), Xantphos (25 mg, 0.043 mmol) and cesium carbonate (0.25 g, 0.77 mmol) in dioxane (10 mL) was heated at reflux under the argon atmosphere for 5 h. After cooling to room temperature, the mixture was filtered and the filtered solid washed with DCM. The filtrate was concentrated and the residue purified flash chromatography on silica gel (30%-50% EtOAc/hexanes) to afford the Boc-protected precursor. To a solution of the Boc-protected precursor in DCM (5 mL) was added TFA (3 mL) and the mixture stirred at RT for 1 h. The reaction was concentrated and purified by HPLC. The fractions were combined and poured into saturated NaHCO₃ solution (30 mL). The combined aqueous layers were extracted with EtOAc (2×30 mL) and the combined organic extracts washed with brine, dried over anhydrous Na₂SO₄ and filtered. The filtrate was concentrated to afford the title compound as a yellow solid (8 mg, 4% in 2 steps).

¹H NMR (500 MHz, DMSO-d₆): δ 2.85-2.90 (m, 4H), 3.05-3.10 (m, 4H), 6.56 (d, J=16.0 Hz, 1H), 6.58 (dd, J=8.1, 2.1 Hz, 1H), 7.02 (dd, J=8.0, 1.5 Hz, 1H), 7.14 (t, J=8.1 Hz, 1H), 7.24-7.29 (m, 2H), 7.32 (s, 1H), 7.53 (d, J=8.7 Hz, 2H), 7.77 (d, J=8.6 Hz, 2H), 7.80 (d, J=7.9 Hz, 1H), 7.97 (d, J=7.8 Hz, 1H), 8.27 (d, J=7.9 Hz, 1H), 8.30 (s, 1H), 10.24 (br s, 1H), 10.52 (s, 1H). MS (ES+): m/z 550 (M+H)⁺.

### Example 67

### (5-Bromo-indazol-1-yl)-phenyl-methanone (Intermediate 43)

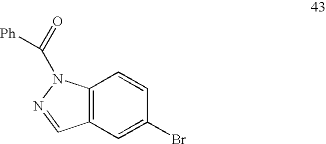

To a solution of 5-bromo-1H-indazole (0.60 g, 3.1 mmol) in dry DCM (15 mL) was added benzoyl chloride (0.40 mL, 3.4 mmol) followed by triethylamine (0.60 mL, 4.3 mmol). The reaction mixture was stirred at room temperature for 16 h and then poured into saturated NaHCO₃ solution (50 mL). The mixture was extracted with EtOAc (2×50 mL) and the combined extracts washed with brine, dried over anhydrous Na₂SO₄ and filtered. The filtrate was concentrated in vacuo and the residue triturated in methanol and the white solid filtered (0.73 g, 80%). MS (ES+): m/z 300 (M+H)⁺.

### Example 68

### 4-(6-{5-[2-(1-Benzoyl-1H-indazol-5-yl)-vinyl]-thiazol-2-ylaminol}-2-methyl-pyrimidin-4-yl)-piperazine-1-carboxylic acid tert-butyl ester (Intermediate 44)

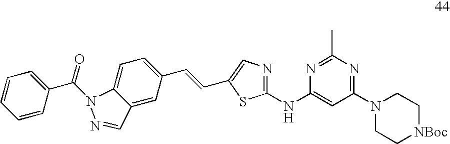

A suspension of intermediate 21 (0.10 g, 0.25 mmol), intermediate 43 (0.10 g, 0.33 mmol), Pd(OAc)₂ (6 mg, 0.027 mmol), PPh₃ (15 mg, 0.057 mmol) and triethylamine (0.10 mL, 0.72 mmol) in DMF (3 mL) was sealed in a microwave reaction tube and irradiated with microwave at 160° C. for 15 min. After cooling down to RT, the cap was removed and the resulting mixture filtered and the filtered solid washed with DCM. The filtrate was concentrated and the residue purified by HPLC. The combined fractions were poured into saturated NaHCO₃ solution (30 mL) and then extracted with EtOAc (2×30 mL). The combined organic extracts were washed with brine, dried over anhydrous Na₂SO₄ and filtered. The filtrate was concentrated to afford the title intermediate as a yellow solid (40 mg, 38%). MS (ES+): m/z 623 (M+H)⁺.

### Example 69

### {5-[2-(1H-Indazol-5-yl)-vinyl]-thiazol-2-yl}-(2-methyl-6-piperazin-1-yl-pyrimidin-4-yl)-amine (Compound XXIV)

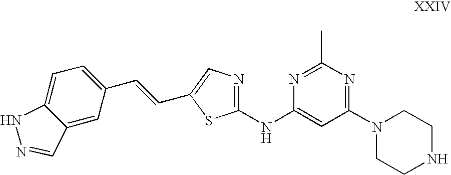

To a solution of intermediate 44 (40 mg, 0.064 mmol) in MeOH (6 mL) was added hydrazine (0.05 mL, 1.6 mmol) and the reaction mixture stirred at room temperature for 3 h. The reaction was concentrated and then suspended in DCM (6 mL). 30% TFA/DCM (6 mL) was added and the mixture stirred at RT for 1 h and concentrated. The residue was purified by HPLC and the combined fractions poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/10). After filtration, the title compound was obtained as a light green solid (10 mg, 30%).

¹H NMR (500 MHz, DMSO-d₆): δ 2.43 (s, 3H), 2.76 (t, J=4.8 Hz, 4H), 3.45 (t, J=4.7 Hz, 4H), 6.05 (s, 1H), 6.86 (d, J=16.2 Hz, 1H), 7.33 (d, J=16.2 Hz, 1H), 7.40 (s, 1H), 7.51 (d, J=8.7 Hz, 1H), 7.64 (dd, J=8.8, 1.4 Hz, 1H), 7.85 (s, 1H), 8.04 (s, 1H), 11.16 (br s, 1H), 13.09 (s, 1H). MS (ES+): m/z 419 (M+H)⁺.

### Example 70

### Determination of Abl and Abl (T3151) Kinase Activity in enzyme assays

Kinase activity was assessed using luminescent detection (KinaseGlo, Promega) of residual ATP concentration in reaction mixtures containing optimized levels of kinase, substrate, ATP, compound and appropriate buffers. Kinase, substrate and ATP concentrations were determined per the supplier's recommendations. Briefly, this involved running concentration curves to select for optimal signal.

Abl Kinase Assay

The reaction mixture (50 μl/well) consisted of Abl kinase (Invitrogen, 20 ng/well); Abltide peptide substrate (Upstate, 100 μM) and ATP (500 nM). Each compound was evaluated at a top concentration of 10 μM with 1:3 dilution steps, 10 dilution steps total. Compound was diluted into DMSO and transferred to the reaction plate, resulting in a final DMSO concentration of 2%. This amount of DMSO was determined not to interfere with the enzyme. The reaction was conducted at 37° C. for 60 minutes. The degree of inhibition was assessed using IC50 determinations, which were obtained using GraphPad Prism4.

Abl (T315I) Kinase Assay

The assay was performed similarly to the Abl kinase assay. Abl (T3151) (Upstate, 17 ng/well); Abltide substrate (100 μM) and ATP (5 nM) comprised the reaction mixture. Compound was similarly diluted, and the reaction plate was incubated for 60 minutes at 37° C. IC50's were calculated as for the Abl kinase assay using GraphPad Prism4.

### Example 71

### Cell Based Assays

Construction of Ba/F3:BCR/ABL Cell Lines:

The human BCR cDNA containing the 3 N-terminal exons and the human ABL cDNA minus the first exon were fused in frame enzymatically and then inserted into a retroviral plasmid vector that carries a neomycin resistant gene (pFB.Neo). The recombinant plasmid was introduced into a retrovirus packaging cell line (EcoPack2-293) through calcium phosphate-mediated transfection to produce replication deficient retrovirus that expresses the BCR/ABL fusion protein. Following the collection of the recombinant retrovirus-containing medium in the transfected EcoPack2-293 cells, a mouse pro-B cell line, Ba/F3, was infected with the recombinant retrovirus. The Ba/F3 cells that have up-taken the recombinant retrovirus and permanently incorporated the viral DNA into the genome were selected by adding G418 to the culture medium at a final concentration of 1 mg/ml. The expression of ˜300 kD BCR/ABL fusion protein was confirmed by Western blot via the presence of the BCR/ABL fusion protein and stimulated phosphorylation of BCR/ABL substrates such as ABL, CrkL and STAT5. To introduce point mutations into the ABL kinase domain of the BCR/ABL fusion protein, site-directed mutagenesis was performed on the recombinant retroviral plasmid vector following the insertion of BCR/ABL fusion cDNA. The introduced point mutations were subsequently confirmed by DNA sequencing prior to transfection in EcoPack2-293 cells.

### Example 72

### Cell Proliferation Assays

Compounds were evaluated for their ability to inhibit the proliferation of BaF3 cells over-expressing the following mutant forms of bcr-abl: no point mutation; T315I, F317L and M351T. Potentially toxic effects were assessed using the parental BaF3 cell line.

Cells were plated at 2500 cells/well. Compounds were pre-diluted at 200× in cell culture medium (10% FBS, Penicillin/Streptomycin/Glutamine in RPMI, plus 10% IL3 for parental cells). The final concentration of DMSO, 0.5%, was determined to not be detrimental to cells. The compound concentration curve was 10 μM top, 1:3 dilution steps, 10 steps. Compound was added immediately after plating the cells, and cells were incubated for 72 hours at 37° C. Cell viability was assessed using XTT (Sigma, 1 mg/ml). IC₅₀ values were determined and normalized as described for the biochemical assays.

The results of the enzymatic assays are shown in Table 1

TABLE 1

| | | | Abl |
|---|---|---|---|
| | | Abl | (T315I) |
| | | IC50 | IC50 |
| Structure | Name | (nM) | (nM) |
| 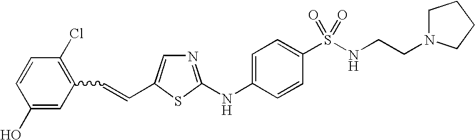 | 4-{5-[2-(2-Chloro-5- hydroxy-phenyl)-vinyl]- thiazol-2-ylamino}-N-(2- pyrrolidin-1-yl-ethyl)- benzenesulfonamide | 890 | |
| 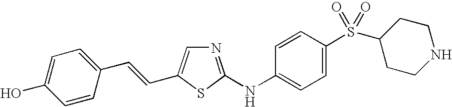 | N2-(4-(2-pyrrolidin-1- yl)ethoxy)phenyl)-5- methyl-N4-p- tolylpyrimidine-2,4- diamine | 215 | 262 |
| 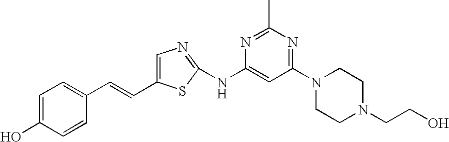 | 4-[2-(2-{6-[4-(2-Hydroxy- ethyl)-piperazin-1-yl]-2- methyl-pyrimidin-4- ylamino}-thiazol-5-yl)- vinyl]-phenol | 26 | 66 |
| 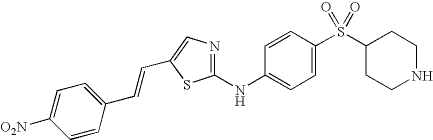 | {5-[2-(4-Nitro-phenyl)- vinyl]-thiazol-2-yl}-[4- (piperidine-4-sulfonyl)- phenyl]-amine | | |
| 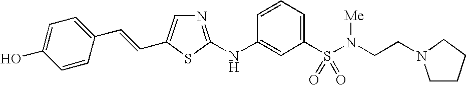 | 3-{5-[2-(4-Hydroxy- phenyl)-vinyl]-thiazol-2- ylamino}-N-methyl-N-(2- pyrrolidin-1-yl-ethyl)- benzenesulfonamide Hydrochloride | 393 | 827 |
| 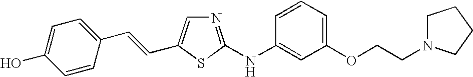 | 4-(2-{2-[3-(2-Pyrrolidin-1- yl-ethoxy)-phenylamino]- thiazol-5-yl}-vinyl)-phenol hydrochloride | 161 | 272 |
| 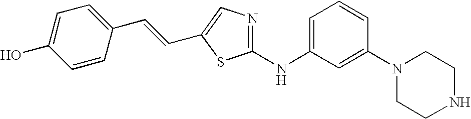 | 4-{2-[2-(3-Piperazin-1-yl- phenylamino)-thiazol-5- yl]-vinyl}-phenol | 24 | 51 |
| 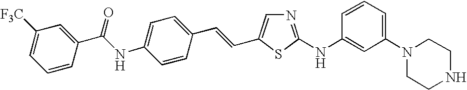 | N-(4-{2-[2-(3-Piperazin-1- yl-phenylamino)-thiazol-5- yl]-vinyl}-phenyl)-3- trifluoromethyl-benzamide | 307 | 7142 |
| 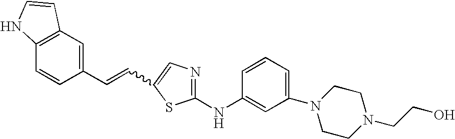 | 2-(4-(3-(5-((E)-2-(1H- indol-5-yl)vinyl)thiazol-2- ylamino)phenyl)piperazin- 1-yl)ethanol Hydrochloride | 174 | 1091 |
| 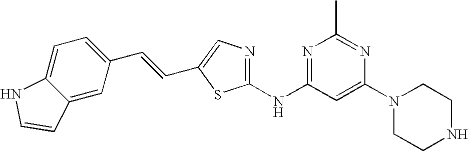 | {5-[2-(1H-Indol-5-yl)- vinyl]-thiazol-2-yl}-(2- methyl-6-piperazin-1-yl- pyrimidin-4-yl)-amine | 54 | 343 |
| 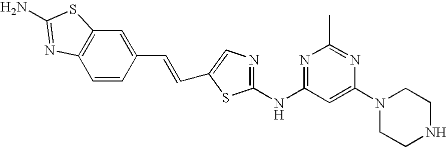 | 6-[(E)-2-{2-[(2-methyl-6- piperazin-1-ylpyrimidin-4- yl)amino]-1,3-thiazol-5- yl}vinyl]-1,3-benzothiazol- 2-amine trifluoroacetic acid salt | 16 | 111 |
| 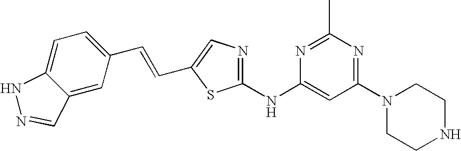 | {5-[2-(1H-Indazol-5-yl)- vinyl]-thiazol-2-yl}-(2- methyl-6-piperazin-1-yl- pyrimidin-4-yl)-amine | 16 | 350 |
| 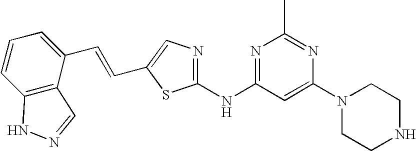 | {5-[2-(1H-Indazol-4-yl)- vinyl]-thiazol-2-yl}-(2- methyl-6-piperazin-1-yl- pyrimidin-4-yl)-amine | 20 | 328 |
| 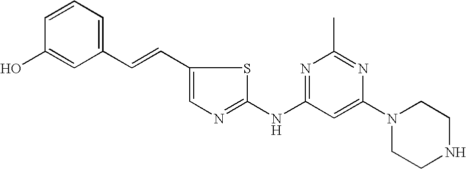 | (E)-3-(2-(2-methyl-6- (piperazin-1-yl)pyrimidin- 4-ylamino)thiazol-5- yl)vinyl)phenol | 1.5 | 18 |
| 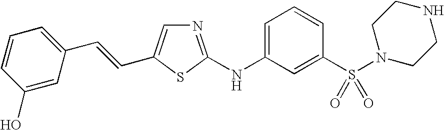 | 3-(-{2-[3-(Piperazine-1- sulfonyl)-phenylamino]- thiazol-5yl}-vinyl)-phenol Hydrochloride | 129 | 2650 |
| 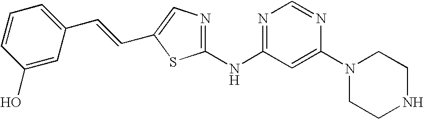 | 3-{2-[2-(6-Piperazin-1-yl- pyrimidin-4-ylamino)- thiazol-5-yl]-vinyl}-phenol | 4.4 | 35 |
| 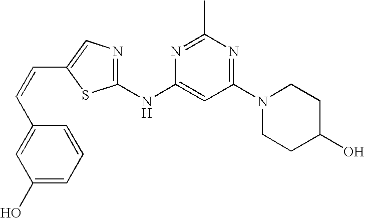 | Cis-1-(6-{5-[2-(3- Hydroxy-phenyl)-vinyl]- thiazol-2-ylamino}-2- methyl-pyrimidin-4-yl)- piperidin-4-ol | 29 | 269 |
| 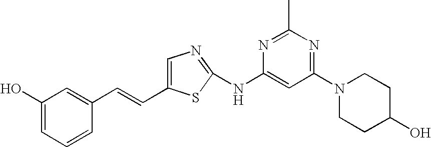 | Trans-1-(6-{5-[2-(3- Hydroxy-phenyl)-vinyl]- thiazol-2-ylamino}-2- methyl-pyrimidin-4-yl)- piperidin-4-ol | 24 | 208 |
| 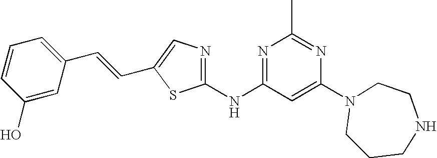 | 3-{2-[2-(6-[1,4]Diazepan- 1-yl-2-methyl-pyrimidin-4- ylamino)-thiazol-5-yl]- vinyl}-phenol | 2.7 | 31 |
| 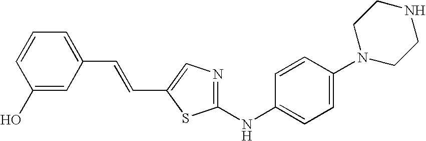 | 2-((E)-2-(2-(4-(piperazin- 1-yl)phenylamino)thiazol- 5-yl)vinyl)phenol Hydrochloride | 15 | 154 |
| 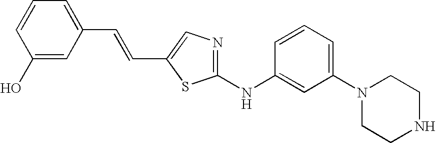 | 3-((E)-2-(2-(3-(piperazin- 1-yl)phenylamino)thiazol- 5-yl)vinyl)phenol Hydrochloride | 14 | 381 |
| 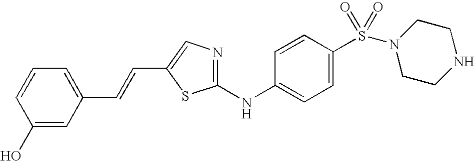 | 3-(-{2-[4-(Piperazine-1- sulfonyl)-phenylamino]- thiazol-5yl}-vinyl)-phenol | 137 | 437 |
| 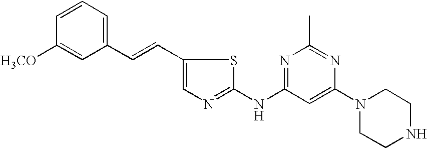 | (E)-5-(3-methoxystyryl)- N-(2-methyl-6-(piperazin- 1-yl)pyrimidin-4- yl)thiazol-2-amine | 224 | 605 |
| 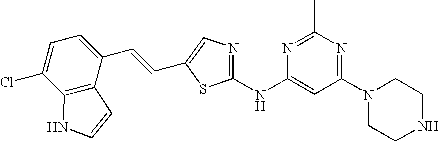 | 4-{5-[2-(3-Hydroxy- phenyl)-vinyl]-pyrimidine- 2-ylamino}-N-piperdin-4- yl-benzenesulfonamide | 70 | 50 |

Although the invention has been described with reference to the above examples, it will be understood that modifications and variations are encompassed within the spirit and scope of the invention. Accordingly, the invention is limited only by the following claims.

What is claimed is:

1. A compound having the general structure (A): 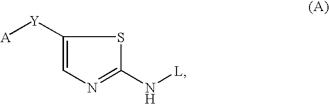
wherein L is a moiety having the structure: 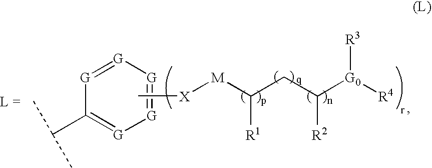
each of the groups G is independently selected from a group consisting of N, CH, or C linked to X, wherein each X is independently selected from a group consisting of, O, C═O, SO₂, or CH₂ and M is a bond, or NR⁹; or X and M taken together is a bond; each of R¹ and R² is independently selected from a group consisting of H, CF₃, F, Cl, Br, I, OH, OCH₃, CN, OCF₃, NH₂, C₁-C₆ substituted or unsubstituted alkyl, substituted or unsubstituted cycloalkyl, substituted or unsubstituted heterocycle, substituted or unsubstituted aryl, or substituted or unsubstituted heteroaryl; or R¹ and R² taken together can be a bond; or R¹ and R² taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ, wherein each of p, q, r, n, m is idependently an integer having the value between 0 and 6.
R⁹ is selected from a group consisting of H, C₁-C₆ substituted or unsubstituted alkyl, C₁ -C₆ substituted or unsubstituted alkenyl, C₁ -C₆ substituted or unsubstituted alkynyl, C₁-C₆ substituted or unsubstituted hydroxyalkyl or aminoalkyl, C₁-C₆ substituted or unsubstituted branched alkyl, C₁-C₆ substituted or unsubstituted cycloalkyl, substituted or unsubstituted aryl connected through carbon or a heteroatom, substituted or unsubstituted heteroaryl connected through carbon or a heteroatom, C_{1-C}₆ alkoxy, a halogen, CF₃, —OCF₃, CHR³R⁴, SR³, SOR³, SO₂R³, SO₂NR³R⁴, SO₃R³, POR³, PO₂R³, PO₂NR³R⁴, PO₂CR³R⁴, PO₃R³, NR³R⁴, NO₂, CN, OH, CONR³R⁴, COR³, COOR³, NR³COR⁴, NR³CONR³R⁴, OCONR³R⁴,CSNR³R⁴, CSR³, NR³CSNR³R⁴, SCONR³R⁴, SCSNR³R⁴, or SCSNR³R⁴;
G₀ is selected from a group consisting of N, O, H, of CH, with the proviso that if G₀ is N, then each of R³ and R⁴ is independently selected from a group consisting of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, or substituted or unsubstituted heteroaryl, or R³ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ;
with additional provisos that if G₀ is N, then R¹ and R⁹ taken together form a moiety selected from a group consisting of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NR⁹—(CH₂)ₘ, or (CH₂)ᵣ—O—(CH₂)ₘ; or R¹ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ; or R³ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ;
with the further proviso that if G₀ is O, then R³ is selected from a group consisting of H, CF₃, F, Cl, Br, I, OH, OCH₃, CN, OCF₃, NH₂, C₁-C₆ alkyl and C₁-C₆ substituted or unsubstituted hydroxyalkyl or aminoalkyl, substituted or unsubstituted branched alkyl, substituted or unsubstituted cycloalkyl, substituted heterocyclic connected through carbon or nitrogen, substituted or unsubstituted aryl, or substituted or unsubstituted heteroaryl connected through carbon or nitrogen, with no group R⁴; R¹ and R⁹ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R¹ and R³ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R³ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ.
with the further proviso that if G₀=CH, then each of R³ and R⁴ is independently selected from a group consisting of H, CF₃, F, Cl, Br, I, OH, OCH₃, CN, OCF₃, NH₂, C₁-₆ alkyl, C₁-₆ substituted or unsubstituted hydroxyalkyl or aminoalkyl, C₁-C₆ substituted or unsubstituted branched alkyl, substituted or unsubstituted aryl, C₁-C₆ substituted or unsubstituted heterocycle connected through carbon or nitrogen, or substituted or unsubstituted heteroaryl connected through carbon or nitrogen, or R³ and R⁴ taken together form a moiety selected from a group consisting of (CHR⁹)ᵣ—(CHR⁹)ₘ—(CHR⁹)ₚ, (CHR⁹)ᵣ—S—(CHR⁹)ₘ, (CHR⁹)ᵣ—SO—(CHR⁹)ₘ, (CHR⁹)ᵣ—SO₂—(CHR⁹)ₘ, (CHR⁹)ᵣ—NR⁹—(CHR⁹)ₘ, and (CHR⁹)ᵣ—O—(CHR⁹)ₘ;
A is an aryl or a heteroaryl moiety selected from a group consisting of: 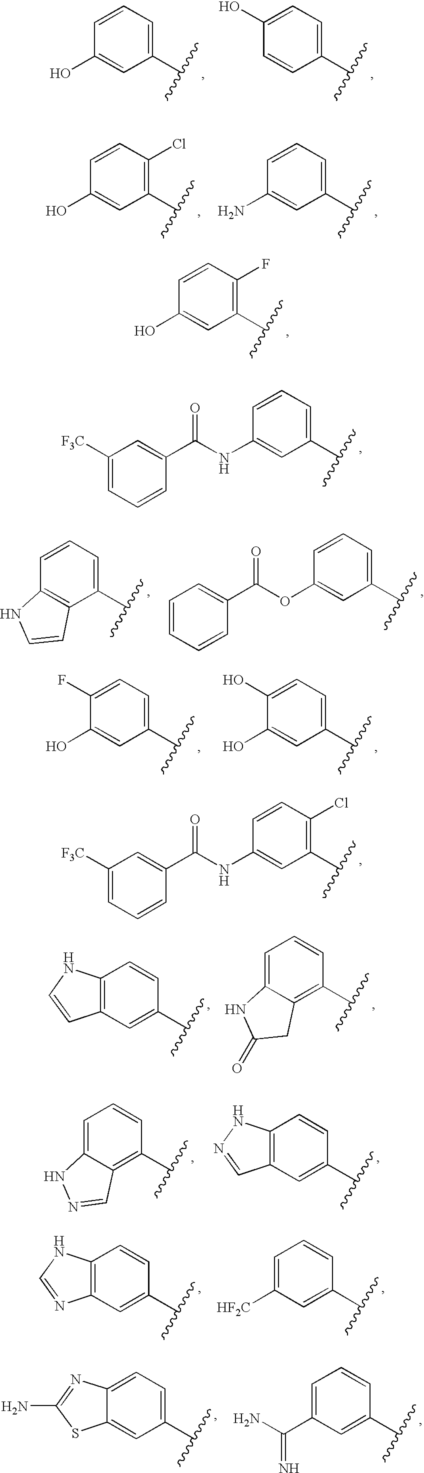 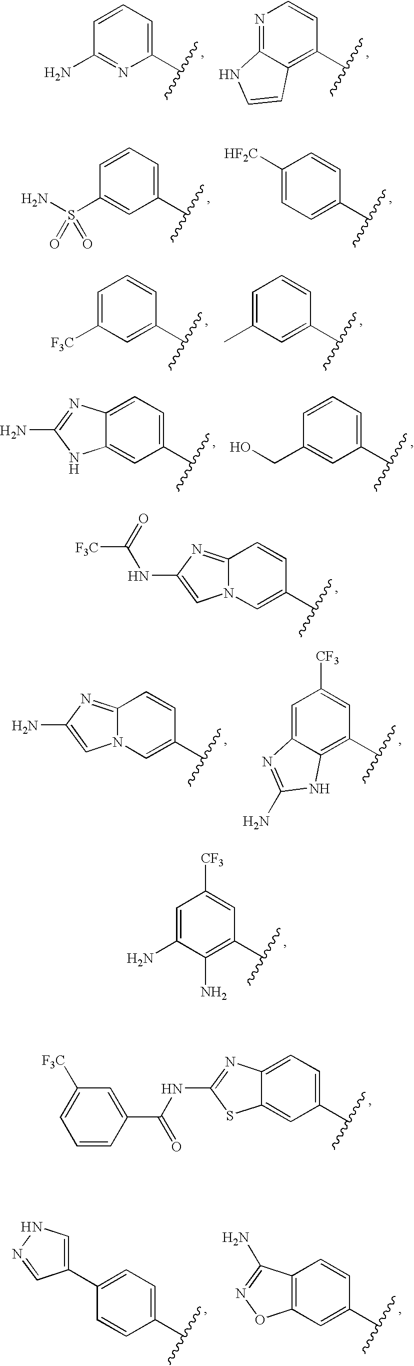 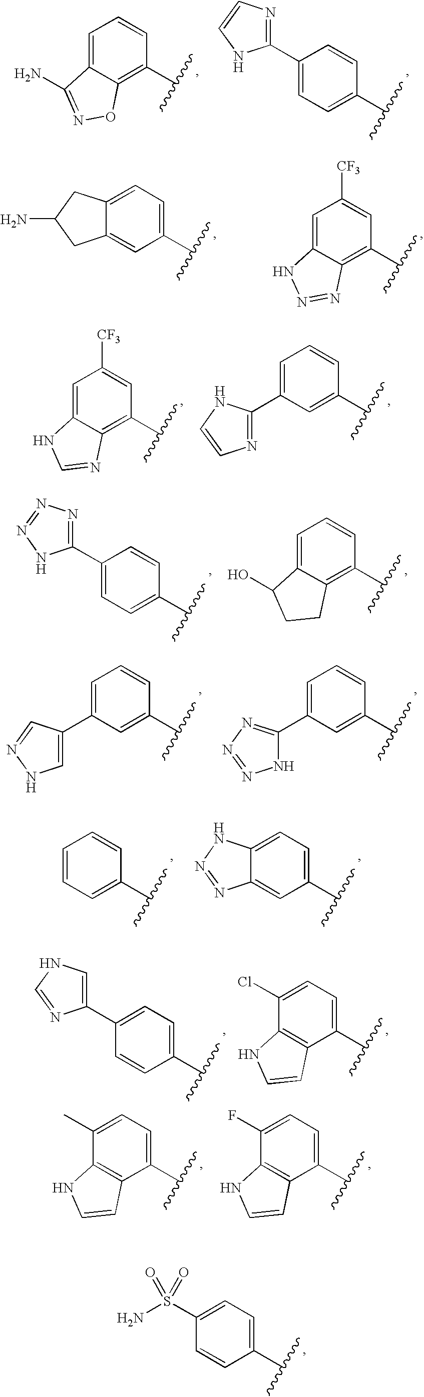 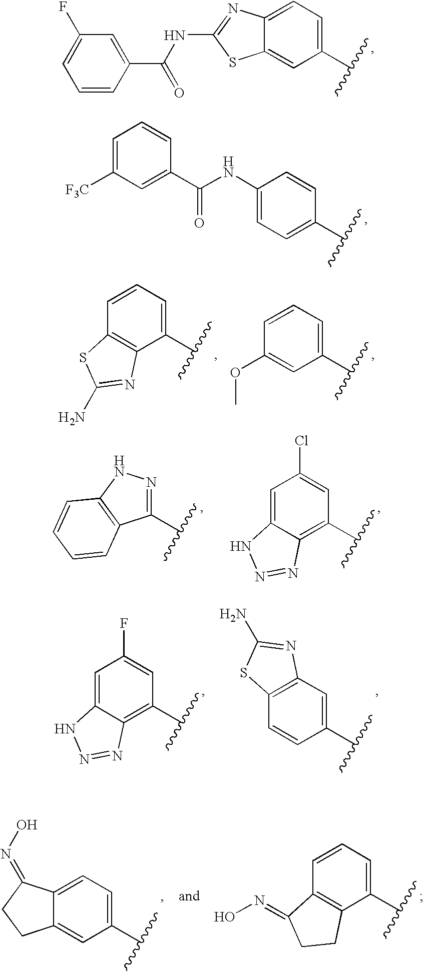
Y is a hydrophobic linking moiety from

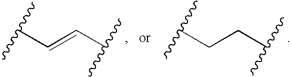

2. A compound of claim 1, wherein L is a substitutent selected from a group consisting of:

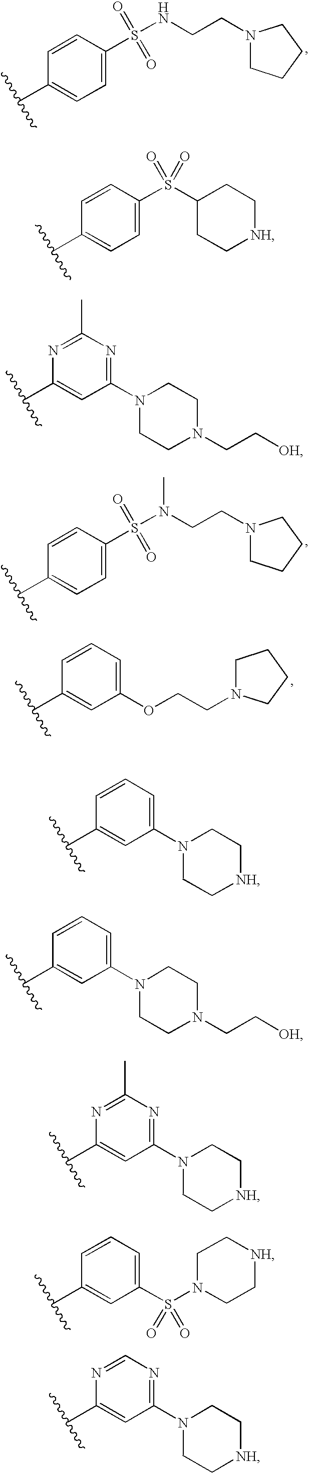

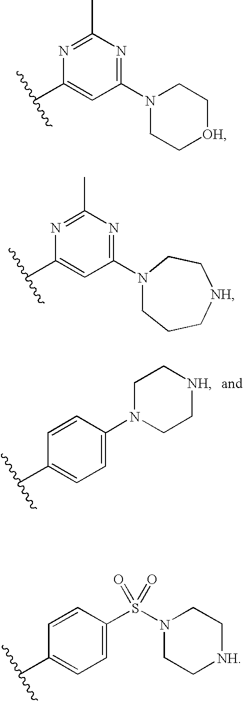

3. The compound of claim 1 or 2, wherein the compound is selected from the group consisting of compounds having formulas (I)-(XXIV):

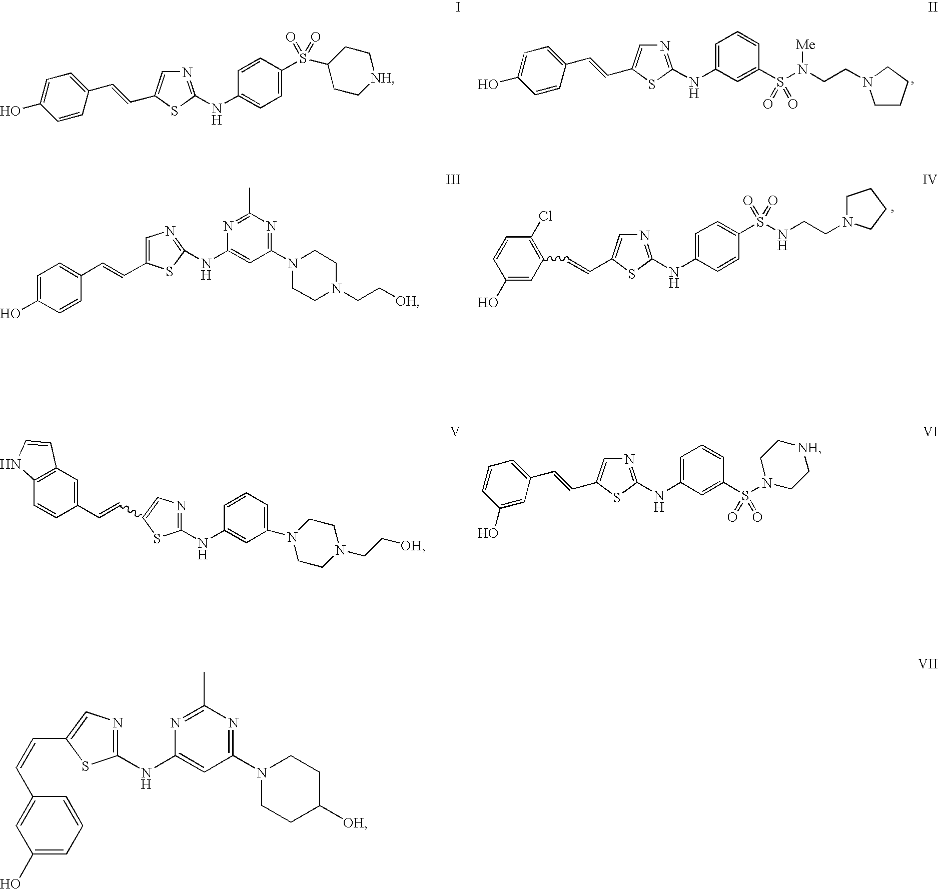

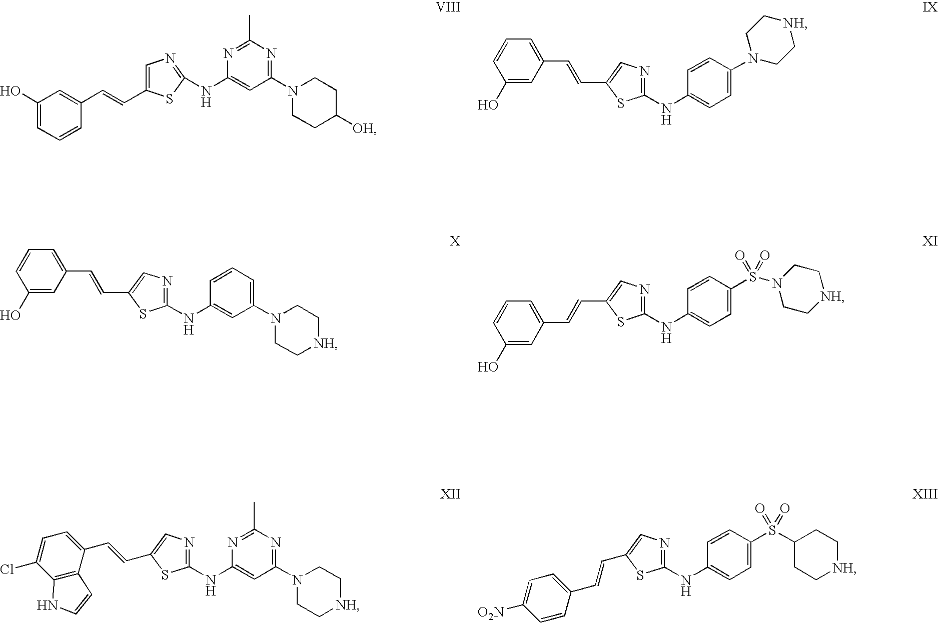

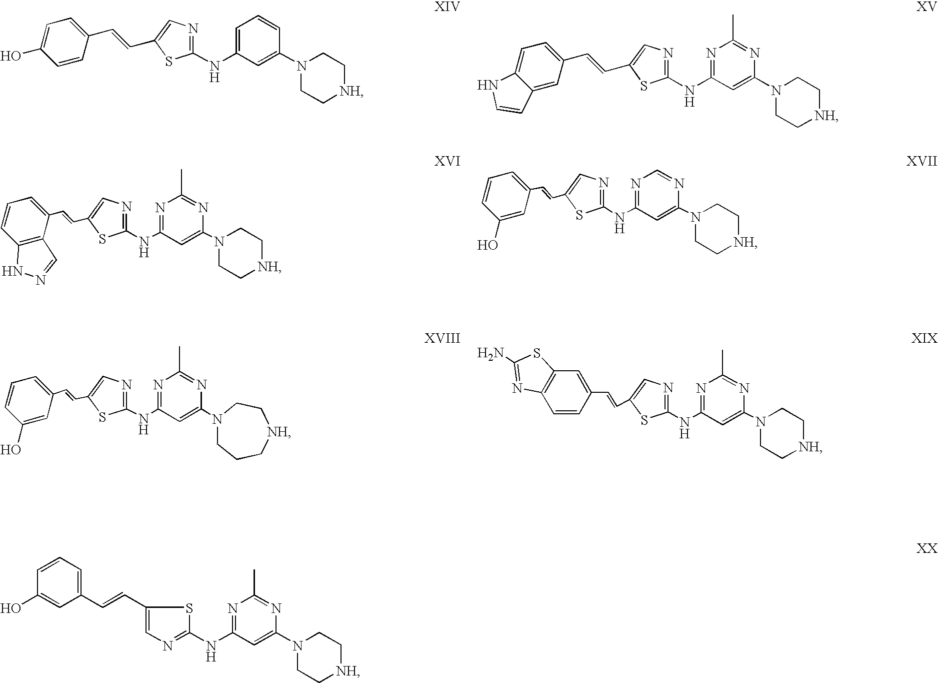

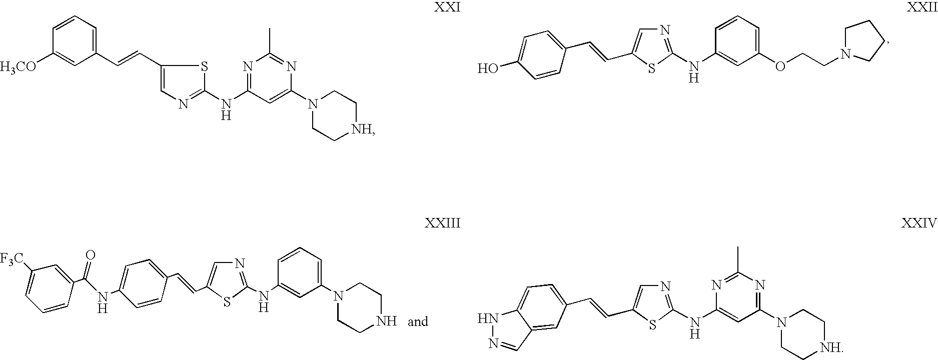

4. The compound of claim 1 or 2, wherein the compound is selected from the group consisting of:

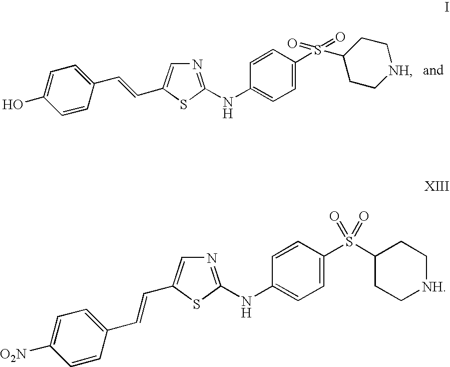

5. The compound of claim 1 or 2, wherein the compound is:

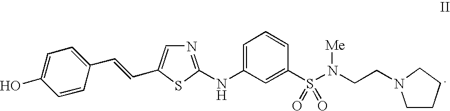

6. The compound of claim 1 or 2, wherein the compound is:

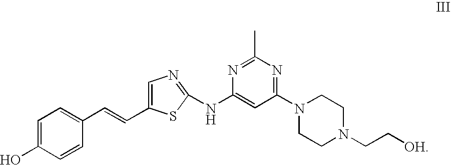

7. The compound of claim 1 or 2, wherein the compound is:

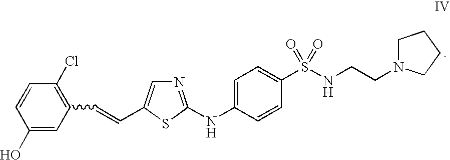

8. The compound of claim 1 or 2, wherein the compound:

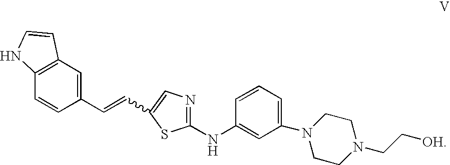

9. The compound of claim 1 or 2, wherein the compound is:

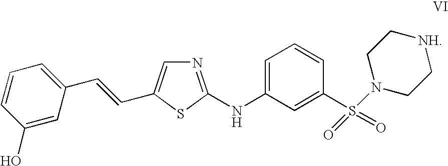

10. The compound of claim 1 or 2, wherein the compound is:

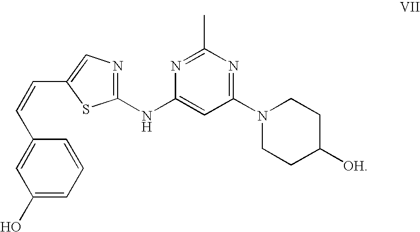

11. The compound of claim 1 or 2, wherein the compound is selected from the group consisting of:

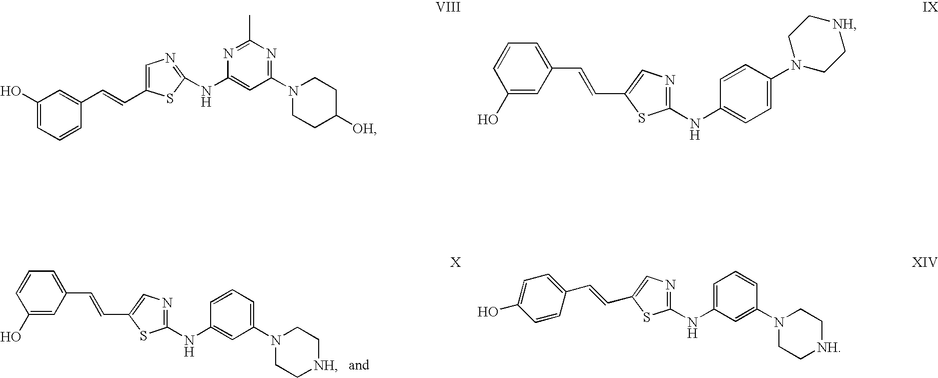

12. The compound of claim 1 or 2, wherein the compound is:

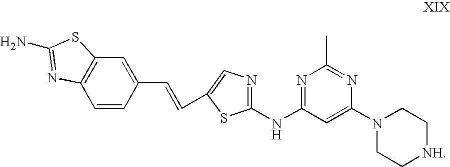

13. The compound of claim 1 or 2, wherein the compound is:

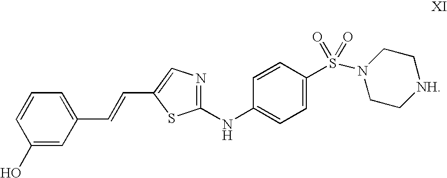

14. The compound of claim 1 or 2, wherein the compound is:

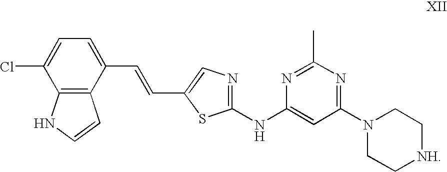

15. The compound of claim 1 or 2, wherein the compound is:

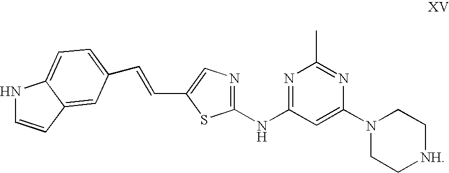

16. The compound of claim 1 or 2, wherein the compound is:

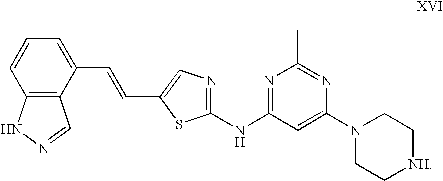

17. The compound of claim 1 or 2, wherein the compound is selected from the group consisting of:

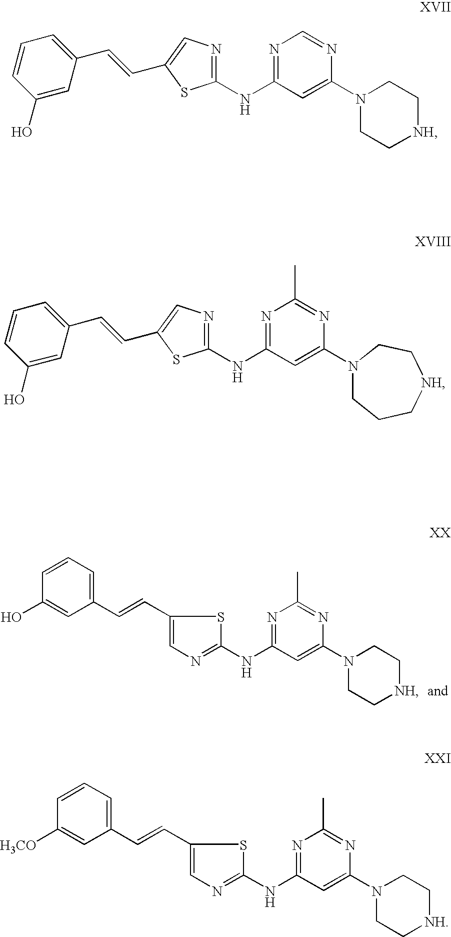

18. The compound of claim 1 or 2, wherein the compound is:

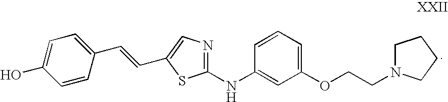

19. The compound of claim 1 or 2, wherein the compound is:

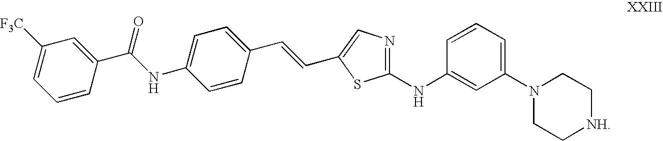

20. The compound of claim 1 or 2, wherein the compound is:

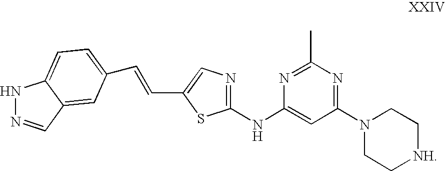

21. A method for treating a disorder, comprising:
(a) in a population of patients in need of the treatment, determining a group of patients who do not respond to any therapy, or any combination of a plurality of therapies, wherein said therapy or therapies comprise administering currently used medications;
(b) administering to a member of the non-responding population a therapeutically effective amount of at least one compound of claim 1 or 2, or pharmaceutically acceptable N-oxide(s), salts, hydrates, solvates, crystal forms and individual diastereomers thereof.

22. The method of claim 21, wherein the currently used medication includes a compound (C), a compound (D), or a compound (E):

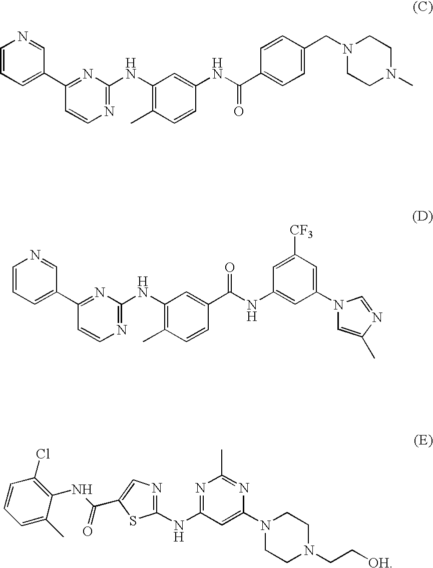

23. The method of claim 21, wherein the non-responsiveness to the kinase-inhibition therapy is caused by the kinase mutation.

24. The method of claim 23, wherein the kinase mutation is the gatekeeper residue mutation.

25. The method of claim 21, wherein the currently used medications comprise GLEEVEC, SPRYCEL, and TASIGNA.

26. The method of claim 25, wherein the currently used medication is GLEEVEC.

27. The method of claim 25, wherein the currently used medication is SPRYCEL.

28. The method of claim 25, wherein the currently used medication is TASIGNA.

29. The method of claim 21, wherein said therapy is a kinase inhibition therapy.

30. The method of claim 21, wherein the disorder is myeloid leukemia in any stage.

31. The method of claim 21, wherein the disorder is an angiogenic disorder.

32. The method of claim 21, wherein the disorder is a hematologic disorder.

33. The method of claim 21, wherein the disorder is a myeloproliferative disorder.

34. The method of claim 21, wherein the disorder is selected from a group consisting of diabetes, a cancer, an eye disease, an inflammation, psoriasis, or a viral infection.

35. The method of claim 34, wherein the cancer is selected from a group consisting of an alimentary/gastrointestinal tract cancer, colon cancer, liver cancer, skin cancer, breast cancer, ovarian cancer, prostate cancer, lymphoma, leukemia, kidney cancer, lung cancer, muscle cancer, bone cancer, bladder cancer and brain cancer.

36. The method of claim 21, wherein the disorder is selected from a group consisting of ocular neovasculariaztion, infantile haemangiomas; organ hypoxia, vascular hyperplasia, organ transplant rejection, lupus, multiple sclerosis, rheumatoid arthritis, psoriasis, Type 1 diabetes and complications from diabetes, inflammatory disease, acute pancreatitis, chronic pancreatitis, asthma, allergies, adult respiratory distress syndrome, cardiovascular disease, liver disease, other blood disorders, asthma, rhinitis, atopic, dermatitits, autoimmune thyroid disorders, ulcerative colitis, Crohn's disease, metastatic melanoma, Kaposi's sarcoma, multiple myeloma, conditions associated with cytokines, and other autoimmune diseases including glomerulonephritis, scleroderma, chronic thyroiditis, Graves' disease, autoimmune gastritis, autoimmune hemolytic anemia, autoimmune neutropenia, thrombocytopenia, allergic asthma, atopic dermatitis, allergic rhinitis, chronic active hepatitis, myasthenia gravis, multiple sclerosis, inflammatory bowel disease, graft vs host disease, motor neuron disease, Alzheimer's disease, Parkinson's disease, amyotrophic lateral sclerosis, Huntington's disease, cerebral ischemia, or neurodegenerative disease caused by traumatic injury, strike, glutamate neurtoxicity, hypoxia; ischemic/reperfusion injury in stroke, myocardial ischemica, renal ischemia, heart attacks, cardiac hypertrophy, atherosclerosis and arteriosclerosis, organ hyoxia, platelet aggregation, allergic contact dermatitis, hypersensitivity pneumonitis, systemic lupus erythematosus, juvenile arthritis, Sjogren's Syndrome, scleroderma, polymyositis, ankylosing spondylitis, psoriatic arthritis, Epstein Barr Virus, Hepatitis B, Hepatitis C, HIV, HTLV1, Vaicella-Zoster Virus, Human Papilloma Virus, food allergy, cutaneous inflammation, and immune suppression induced by solid tumors.

37. The method of claim 21, wherein the disorder is associated with a kinase.

38. The method of claim 21, wherein the disorder is associated with gatekeeper mutations in the kinase.

39. A pharmaceutical composition comprising at least one compound of claim 1 or 2, or pharmaceutically acceptable N-oxide(s), salts, hydrates, solvates, crystal forms and individual diastereomers thereof, and a pharmaceutically acceptable carrier therefore.

40. An article of manufacture comprising packaging material and a pharmaceutical composition contained within the packaging material, wherein the packaging material comprises a label which indicates that the pharmaceutical composition can be used for treatment of angiogenic-associated disorders, and wherein the pharmaceutical composition comprises at least one compound of claim 1 or 2, or pharmaceutically acceptable N-oxide(s), salts, hydrates, solvates, crystal forms and individual diastereomers thereof.

41. An article of manufacture comprising packaging material and a pharmaceutical composition contained within the packaging material, wherein the packaging material comprises a label which indicates that the pharmaceutical composition can be used for treatment of myeloproliferative disorder, proliferative diabetic retinopathy, a cancer, eye disease, inflammation, psoriasis, or a viral infection, and wherein the pharmaceutical composition comprises at least one compound of claim 1 or 2, or pharmaceutically acceptable N-oxide(s), salts, hydrates, solvates, crystal forms and individual diastereomers thereof.

42. The article of manufacture of claim 40, wherein the disorder is selected from a group consisting of an alimentary/gastrointestinal tract cancer, colon cancer, liver cancer, skin cancer, breast cancer, ovarian cancer, prostate cancer, lymphoma, leukemia, kidney cancer, lung cancer, muscle cancer, bone cancer, bladder cancer and brain cancer.

43. A method for reducing or eliminating resistance of a protein associated with a disorder, to currently used therapies, comprising synthesizing a compound of claim 1 or 2, wherein said compound is effective as an inhibitor of said protein, thereby overcoming said resistance.
